(19) United States
(12) Patent Application Publication
(10) Pub. No.: US 2011/0224182 A1
(43) Pub. Date: Sep. 15, 2011

(54) SALTS OF (3-0-(3',3'-DIMETHYLSUCCINYL) BETULINIC ACID AND SOLID STATE FORMS THEREOF

(75) Inventors: Peter Bullock, Fitchburg, WI (US); Theodore J. Nitz, Boyds, MD (US); Gary Sweetapple, Fairport Harbor, OH (US)

(73) Assignee: Myriad Pharmaceuticals, Incorporated, Salt Lake City, UT (US)

(21) Appl. No.: 12/504,883

(22) Filed: Jul. 17, 2009

Related U.S. Application Data
(63) Continuation of application No. PCT/US2008/000635, filed on Jan. 18, 2008.
(60) Provisional application No. 60/881,195, filed on Jan. 19, 2007.

(51) Int. Cl. A61K 31/56; C07J 53/00; A61P 31/18

(52) U.S. Cl. 514/182; 552/510

(57) ABSTRACT

The present invention concerns novel pharmaceutically active compounds, pharmaceutical compositions containing the same, methods of making the compounds, polymorphic forms of the compounds, the compounds for use as medicaments, and use of the compounds for the manufacture of medicaments. The present invention also concerns a method of treatment involving administration of the compounds. Specifically, the compounds are certain salts of 3-O-(3′,3′-dimethylsuccinyl)betulinic acid, also known as “DSB.” The novel compounds are useful as antiretroviral agents. In particular, the novel compounds are useful for the treatment of Human Immunodeficiency Virus (“HIV”).

### CROSS REFERENCE TO RELATED APPLICATIONS

This application is a continuation of International Application PCT/US2008/000635, filed Jan. 18, 2008, which claims priority to U.S. Provisional Application Ser. No. 60/881,195 filed on Jan. 19, 2007, both of which are incorporated herein by reference in their entirety.

### BACKGROUND OF THE INVENTION

1. Field of the Invention

The present invention concerns novel pharmaceutically active compounds, pharmaceutical compositions containing the same, methods of making the compounds, polymorphic forms of the compounds, the compounds for use as medicaments, and use of the compounds for the manufacture of medicaments. The present invention also concerns a method of treatment involving administration of the compounds. Specifically, the compounds are certain salts of 3-O-(3′,3′-dimethylsuccinyl)betulinic acid, also known as “DSB.”

The novel compounds are useful as antiretroviral agents. In particular, the novel compounds are useful for the treatment of Human Immunodeficiency Virus (“HIV”).

2. Related References

HIV is a member of the lentiviruses, a subfamily of retroviruses. HIV infects and invades cells of the immune system; it breaks down the body's immune system and renders the patient susceptible to opportunistic infections and neoplasms. The immune defect appears to be progressive and irreversible, with a high mortality rate.

U.S. Pat. No. 5,679,828 mentions betulinic acid and dihydrobetulinic acid derivatives, including 3-O-(3′,3′-dimethylsuccinyl)betulinic acid (“DSB”) (structure shown below) as potent anti-HIV agents.

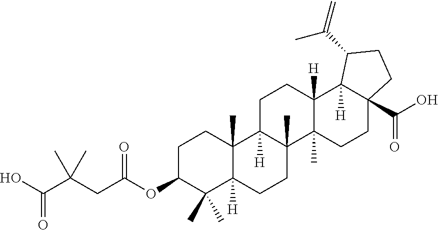

U.S. Published Patent Application No. 2005/0239748 mentions N-methylglucamine (NMG), potassium, and sodium salts of DSB as compounds that are useful in the treatment of HIV and related diseases.

It is well known in the art that highly water soluble medicinal preparations, when administered orally, result in efficient absorption of such preparations from the gastrointestinal tract into systemic circulation. Another hallmark of such preparations is the rapid rate at which they are absorbed into the systemic circulation resulting in a high concentration of the active agent in the blood.

Despite recent progress in the development of anti-HIV therapeutic options, there remains a need for drugs having different or enhanced anti-HIV properties relative to currently marketed pharmaceuticals.

DSB is a hydrophobic molecule exhibiting high molecular weight, high log P, and low aqueous solubility. The identification of counterions that (i) are capable of forming a stable crystalline form with DSB; and (ii) demonstrate at least one superior pharmacokinetic or pharmacodynamic property, including enhanced solubility, enhanced absorption, improved bioavailability, or greater Cₘₐₓ, would satisfy a long felt need in this art.

The identification of an appropriate counterion from which a DSB salt could be synthesized via a robust route suitable for large scale manufacturing would satisfy an additional long felt need in this art.

A need continues to exist for novel compounds which possess potent antiretroviral activity, especially anti-HIV activity, with improved pharmacokinetic and pharmacodynamic properties. A further need exists for methods of synthesizing novel compounds which possess potent antiretroviral activity, especially anti-HIV activity, with improved pharmacokinetic and pharmacodynamic properties. A further need exists for methods of treating HIV infected patients with novel compounds which possess potent antiretroviral activity, especially anti-HIV activity, with improved pharmacokinetic and pharmacodynamic properties.

### BRIEF SUMMARY OF THE INVENTION

There is now provided a salt form of DSB comprising DSB and (+)-arginine in a 1:2 ratio (DSB-2 (+)-arginine). In some embodiments, the (+)-arginine disalt of DSB is amorphous. In some embodiments, the (+)-arginine disalts of DSB are crystalline. In some embodiments, the crystalline (+)-arginine disalts of DSB are polymorphic Form I-a. In some embodiments, the crystalline (+)-arginine disalts of DSB are polymorphic Form II-a.

There is now further provided a salt form of DSB comprising DSB and choline in a 1:2 ratio (DSB-2 choline). In some embodiments, the choline disalt of DSB is amorphous. In some embodiments, the choline disalts of DSB are crystalline. In some embodiments, the crystalline choline disalts of DSB are polymorphic Form I-c. In some embodiments, the crystalline choline disalts of DSB are polymorphic Form II-c. In some embodiments, the crystalline choline disalts of DSB are polymorphic Form III-c.

There is now further provided a salt form of DSB comprising DSB and diethanolamine in a 1:2 ratio (DSB-2 diethanolamine). In some embodiments, the diethanolamine disalts of DSB are crystalline. In some embodiments, the crystalline diethanolamine disalts of DSB are polymorphic Form I-o. In some embodiments, the crystalline diethanolamine disalts of DSB are polymorphic Form II-o.

There is now further provided a salt form of DSB comprising DSB and diethylamine in a 1:2 ratio (DSB-2 diethylamine). In some embodiments, the diethylamine disalts of DSB are crystalline. In some embodiments, the crystalline diethylamine disalts of DSB are polymorphic Form I-y. In some embodiments, the crystalline diethylamine disalts of DSB are polymorphic Form II-y. In some embodiments, the crystalline diethylamine disalts of DSB are polymorphic Form III-y. In some embodiments, the crystalline diethylamine disalts of DSB are polymorphic Form IV-y.

There is now further provided a pharmaceutical composition comprising a disalt of DSB and a pharmaceutically acceptable excipient.

There is now further provided a method of using a pharmaceutical composition comprising a disalt of DSB and a pharmaceutically acceptable excipient.

There is now further provided a method of synthesizing a disalt of DSB. There is now further provided a method of synthesizing an amorphous disalt of DSB. There is now further provided a method of synthesizing a crystalline disalt of DSB.

There is now further provided a method of synthesizing a pharmaceutical composition comprising a disalt of DSB and a pharmaceutically acceptable excipient.

These compositions and dosage forms can be used in methods of treating HIV and related diseases.

Methods of making the salts of DSB and the pharmaceutical compositions are also provided.

### BRIEF DESCRIPTION OF THE DRAWINGS

FIG. 1 depicts a powder x-ray diffraction (XRD) of DSB free acid.

FIG. 2 depicts a differential scanning calorimetry-thermogravimetric analysis overlay plot of DSB free acid.

FIG. 3 depicts a differential scanning calorimetry-thermogravimetric analysis overlay plot of DSB (+)-arginine disalt.

FIG. 4 depicts a powder XRD of DSB (+)-arginine disalt Form I-a.

FIG. 5 depicts a ¹H FT-NMR spectrum of DSB (+)-arginine disalt.

FIG. 6 depicts a powder XRD of DSB (+)-arginine disalt Form II-a.

FIG. 7 depicts a differential scanning calorimetry-thermogravimetric analysis overlay plot of DSB choline disalt.

FIG. 8 depicts a powder XRD of DSB choline disalt Form I-c.

FIG. 9 depicts a ¹H FT-NMR spectrum of DSB choline disalt.

FIG. 10 depicts a powder XRD of DSB choline disalt Form I-c.

FIG. 11 depicts a powder XRD of DSB choline disalt Form II-c.

FIG. 12 depicts a powder XRD of DSB choline disalt Form III-c.

FIG. 13 depicts a powder XRD of DSB choline disalt Form IV-c.

FIG. 14 depicts a differential scanning calorimetry-thermogravimetric analysis overlay plot of DSB diethanolamine disalt.

FIG. 15 depicts a powder XRD of DSB diethanolamine disalt Form I-o.

FIG. 16 depicts a ¹H FT-NMR spectrum of DSB diethanolamine disalt.

FIG. 17 depicts a powder XRD of DSB diethanolamine disalt Form II-o.

FIG. 18 depicts a powder XRD of DSB diethanolamine disalt Form III-o.

FIG. 19 depicts a differential scanning calorimetry-thermogravimetric analysis overlay plot of DSB diethylamine disalt.

FIG. 20 depicts a powder XRD of DSB diethylamine disalt Form I-y.

FIG. 21 depicts a ¹H FT-NMR spectrum of DSB diethylamine disalt.

FIG. 22 depicts a powder XRD of DSB diethylamine disalt Form I-y.

FIG. 23 depicts a powder XRD of DSB diethylamine disalt Form IV-y.

FIG. 24 depicts a powder XRD of DSB diethylamine disalt Form II-y.

FIG. 25 depicts a powder XRD of DSB diethylamine disalt Form III-y.

FIG. 26 depicts a graph of individual DSB-2 NMG plasma concentrations of Group 1 rats vs. time following intravenous administration.

FIG. 27 depicts a graph of mean DSB-2 NMG plasma concentrations of Group 1 rats vs. time following intravenous administration.

FIG. 28 depicts a graph of individual DSB-2 NMG plasma concentrations of Group 2 rats vs. time following oral gavage administration.

FIG. 29 depicts a graph of mean DSB-2 NMG plasma concentrations of Group 2 rats vs. time following oral gavage administration.

FIG. 30 depicts a graph of individual DSB-2 (+)-arginine plasma concentrations of Group 3 rats vs. time following oral gavage administration.

FIG. 31 depicts a graph of mean DSB-2 (+)-arginine plasma concentrations of Group 3 rats vs. time following oral gavage administration.

FIG. 32 depicts a graph of individual DSB-2 choline plasma concentrations of Group 4 rats vs. time following oral gavage administration.

FIG. 33 depicts a graph of mean DSB-2 choline plasma concentrations of Group 4 rats vs. time following oral gavage administration.

FIG. 34 depicts a graph of individual DSB-2 diethanolamine plasma concentrations of Group 5 rats vs. time following oral gavage administration.

FIG. 35 depicts a graph of mean DSB-2 diethanolamine plasma concentrations of Group 5 rats vs. time following oral gavage administration.

FIG. 36 depicts a graph of individual DSB-2 diethylamine plasma concentrations of Group 6 rats vs. time following oral gavage administration.

FIG. 37 depicts a graph of mean DSB-2 diethylamine plasma concentrations of Group 6 rats vs. time following oral gavage administration.

### DETAILED DESCRIPTION OF THE INVENTION

The compounds of the present invention have utility in anti-retroviral applications, especially anti-HIV applications. The treatment of HIV is a preferred use. All forms of HIV are potentially treatable with compounds of the present invention. Compounds of the present invention have utility in treating protease inhibitor resistant HIV, reverse transcriptase inhibitor resistant HIV, and entry or fusion inhibitor resistant HIV. Compounds of the present invention have utility in treating HIV-1, including subtypes A1, A2, B, C, D, F1, F2, G, H, J and K; and circulating recombinant HIV forms. Compounds of the present invention have utility in treating HIV groups M, N, and O. Compounds of the present invention have utility in treating HIV strains capable of binding to the human CCR5 receptor and HIV strains capable of binding to the human CXCR4 receptor.

The compounds of the present invention have utility in anti-neoplastic applications; all forms of neoplasia are potentially treatable with compounds of the present invention. Compounds of the present invention have utility in treating brain cancer; bone cancer; leukemias; lymphomas; epithelial cell-derived neoplasias or epithelial carcinomas including basal cell carcinoma; adenocarcinoma; gastrointestinal cancers including lip cancers, mouth cancers, esophogeal cancers, small bowel cancers and stomach cancers; colon cancers; liver cancers; bladder cancers; pancreatic cancers; ovary cancers; cervical cancers; lung cancers; breast cancers; and, skin cancers including as squamous cell cancers and basal cell cancers; prostate cancers; renal cell carcinomas; and other known cancers effecting epithelial cells.

Without wishing to be bound by theory, the DSB salts of the present invention inhibit cleavage of the capsid-SP1 polyprotein resulting in the release of virion-like particles incapable of maturing into an infectious virion.

The term “about” means refers to the normal variation in that measured quantity, as expected by the skilled artisan making the measurement and exercising a level of care commensurate with the objective of measurement and the precision of the measuring equipment. When used in relation with amount of time, “about” can have its ordinary meaning, and can be used to round the amount of time to simplify the language, for example, “about a few days” rather than “60 hours”.

The term “amorphous” means a solid state form wherein the molecules are present in a disordered arrangement and do not form a distinguishable crystal lattice or unit cell. When subjected to X-ray powder diffraction, amorphous compounds do not produce a diffraction pattern characteristic of a crystalline form.

The term “anti-retroviral activity” or “anti-HIV activity” means the ability to inhibit at least one of:

(1) retroviral attachment to cells;

(2) viral entry into cells;

(3) viral pro-DNA integration into host cell genome;

(4) cellular metabolism which permits viral replication;

(5) inhibition of intercellular spread of the virus;

(6) synthesis of viral antigens;

(7) cellular expression of viral antigens

(8) viral budding or maturation;

(9) activity of virus-coded enzymes (such as reverse transcriptase, integrase and proteases); or

(10) any known retroviral or HIV pathogenic actions, such as, for example, immunosuppression.

The term “AUC” means the area under the concentration:time curve.

The term “Cₘₐₓ” means the maximum serum concentration of a compound. The term “crystalline form” means a solid state form wherein the molecules are arranged to form a distinguishable crystal lattice (i) comprising distinguishable unit cells, and (ii) yielding diffraction peaks when subjected to X-ray radiation.

The term “bioavailability” means the rate and extent to which the active ingredient is absorbed from a drug product and becomes available at the site of action as detailed in the Code of Federal Regulations, Title 21, Part 320.1. Bioavailability data for a particular compound and formulation provides an estimate of the fraction of the administered dose, for example, an oral tablet, that is absorbed into the systemic circulation.

The term “DMEM” means Dulbecco's Modified Eagle Medium.

The term “drug substance” as used herein means DSB di-salt per se as qualified by the context in which the term is used, and can refer to an unformulated DSB di-salt or to a DSB di-salt present as an ingredient of a pharmaceutical composition.

The term “EC₅₀” means the drug concentration that results in a 50% reduction in virus replication.

The term “free drug” means the unbound drug fraction of the bound-to-total concentration ratio in whole blood or plasma.

The term “phase pure” means purity with respect to other solid state forms of a DSB di-salt and does not necessarily imply a high degree of chemical purity with respect to other compounds.

The term “therapeutic effect” means some extent of relief of one or more of the symptoms of an HIV related disorder. In reference to the treatment of HIV, a therapeutic effect refers to one or more of the following: 1) reduction in the number of infected cells; 2) reduction in the number of virions present in serum; 3) inhibition (i.e., slowing to some extent, preferably stopping) of rate of HIV replication; 4) relieving or reducing to some extent one or more of the symptoms associated with HIV; and 5) relieving or reducing the side effects associated with the administration of other anti-retroviral agents.

The term “therapeutically effective amount” means the amount required to achieve a therapeutic effect.

The term “Tₘₐₓ” means the time required to reach the maximum serum concentration of a compound.

The term “weight percent” means the weight percent of a specified ingredient based upon the total weight of all ingredients of the composition.

The term “without food” means the condition of not having consumed food during the period between from at least about 12 hours prior to the administration of a compound to at least about 4 hours after the administration of the compound with water being made available ad libitum.

In an effort to improve pharmacokinetic and pharmacodynamic profiles of DSB, a number of pharmaceutically acceptable bases were evaluated to determine which bases might have appropriate ionization centers, pKₐ values, and molecular weight. Of these bases, ammonium hydroxide, (+)-arginine, (−)-arginine, choline hydroxide, diethanolamine, diethylamine, (+)-lysine, magnesium hydroxide, potassium hydroxide, sodium hydroxide, triethanolamine, and tris(hydroxymethyl)aminomethane were selected as reagents in the synthesis of twenty four salt forms of DSB. Thirteen solvents were employed to assess the potential for polymorphism of the solid state salt forms of DSB. Of the possible 156 base:solvent combinations, five (5) disalts have been identified as markedly superior relative to the DSB free acid with respect to at least one pharmaceutically significant property. (+)-Arginine, choline, diethanolamine, and diethylamine disalt forms of DSB exhibit one or more of the following superior properties thereby satisfying a long felt need in the art of virology and augmenting pharmaceutical options for clinicians providing anti-retroviral treatment to those in need thereof. These properties include, but are not limited to, one or more of the following:

(1) improved bioavailability;

(2) improved solubility of the pharmaceutical composition;

(3) reduced moisture content or hygroscopicity of oral dosage forms;

(4) improved safety for oral dosage forms;

(5) improved composition wettability;

(6) improved particle size distribution;

(7) improved composition compressibility; and

(8) improved composition flow properties.

Each of the (+)-arginine, choline, diethanolamine, and diethylamine disalt forms were studied to identify polymorphs that further enhance one or more of the properties listed above. Recrystallization of solids resulting from the initial salt synthesis is a preferred method of synthesizing such polymorphs. For each disalt at least one suitable recrystallization solvent was identified. A recrystallization solvent was considered suitable if: the DSB disalt was very soluble near the boiling point of the recrystallization solvent and at most sparingly soluble at reduced temperatures, for example room temperature; some or all impurities, if present, are soluble in the recrystallization solvent at reduced temperatures; and the recrystallization solvent does not react with the DSB disalt.

Certain crystalline forms and amorphous forms of (+)-arginine disalts and choline disalts are presently disclosed. Certain crystalline forms of diethanolamine disalts, and diethylamine disalts are presently disclosed. As the crystalline disalts of DSB exhibit greater chemical and physical stability relative to both the zwitterion and the amorphous salt form, crystalline disalts of DSB are superior to both the zwitterion and the amorphous salt form for the preparation of solid pharmaceutical dosage forms.

### Method A—Molecular Spectroscopy—1H-NMR

Samples were prepared by dissolving about 1 mg to about 3 mg in dimethylsulfoxide

(DMSO)-d₆, methanol-d₄, or THF-dₛ with 0.05% (v/v) tetramethylsilane (TMS). Spectra were collected at ambient temperature on a Varian Gemini 300 MHz FT-NMR spectrometer.

### Method B—Solubility

Measurement of the water solubility of the salts of this invention is accomplished by using methods well known to those skilled in the art. Specifically, to a weighed amount of DSB (+)-arginine disalt, DSB choline disalt, DSB diethanolamine disalt, or DSB diethylamine disalt, a solvent is added in small portions until a clear solution is obtained. The total volume of the solution is measured. The solubility of the particular DSB salt, in mg/mL, is calculated by dividing the weight of the salt (in mg), by the volume of the solution (in mL). The solubility of each DSB disalt was determined at ambient temperature in a plurality of solvents including: water, methanol, ethanol, 2,2,2-trifluoroethanol, 1-propanol, and 2-propanol.

### Method C—Differential Scanning Calorimetry (DSC)

Samples were prepared by crimping about 1 mg to about 10 mg in aluminum sample pans and scanned from 25° C. to about 300° C. at 10° C./minute using a nitrogen purge at 50 mL/min. DSC data were collected on a TA Instruments 2910 Differential Scanning calorimeter.

### Method D—Thermogravimetric Analysis (TGA)

Samples were prepared by placing about 5 mg to about 15 mg in an open, pre-tared platinum sample pan and scanned from 25° C. to about 250° C. at 10° C./minute using a nitrogen purge. TGA data were collected on a TA Instruments 2950 Thermogravimetric Analyzer.

### Method E—Hot Stage Microscopy (HSM)

Samples were prepared by mounting a specimen on a microscope slide with a drop of immersion oil and a cover glass. A Zeiss Universal microscope configured with a polarized visible light source and a Mettler hot stage accessory was used. Magnification was typically 250×. Samples were heated from 25° C. to about 300° C. at 3° C./minute or 10° C./minute. Physical observations including phase change, recrystallization, and evolution of bubbles was recorded where applicable.

### Method F—Microscopy

Samples were prepared by mounting a specimen on a microscope slide with a drop of immersion oil and a cover glass. A Zeiss Universal microscope configured with a polarized visible light source was used to evaluate the optical properties of the samples. Magnification was typically 250×. Physical observations including particle size, crystal size, crystal shape, and the presence of birefringence were recorded where applicable.

### Method G—Powder X-Ray Diffraction (PXRD)

Samples were mounted in low background quartz plates (9 mm diameter, 0.2 mm deep cavity). Diffraction patterns were collected using a Bruker D8 Discovery diffractometer configured with an XYZ stage, laser video microscope for positioning, and HiStar area detector. Collection times were 60 seconds at room temperature. A Cu—K α radiation 1.5406 Å tube was operated at 40 kV and 40 mA. The X-ray optics consist of a Gobel mirror coupled with a pinhole collimator of 0.5 mm. Theta-theta continuous scans were employed with a sample-detector distance of 15 cm, which gives an effective 2θ range of 4-40° C.

### Method H—In Vivo Pharmacokinetic Studies

In vivo experiments were conducted in male Sprague-Dawley rats having a body mass of 225 g to 275 g with an appropriate cannula surgically inserted. Rats were supplied food and water ad libitum until 12 hours before dosing when food was removed. Food was withheld until four hours post-dosing. Water was available ad libitum. Each of group of rats consisted of 3 rats.

Dosing solutions were prepared the same day as dose administration. The formulation vehicle was 3% DMA and a 97% solution of 20% hydroxypropyl-β-cyclodextrin in water. The administered dose of each salt was 1 mg/kg for intravenous administration and 10 mg/kg for oral gavage. Formulations for intravenous administration were filtered after preparation. Concentrations of respective dosing solutions were as depicted in Table 1 below.

TABLE 1
Concentrations of Dosing Solution

| | |
|---|---|
| Dosing | Conc. (mg/mL) |

| Solution | Rep. 1 | Rep. 2 | Mean |
|---|---|---|---|
| Group 1 | 0.96 | 0.97 | 0.97 |
| Group 2 | 1.94 | 1.87 | 1.91 |
| Group 3 | 1.95 | 2.08 | 2.02 |
| Group 4 | 1.88 | 2.12 | 2.00 |
| Group 5 | 2.34 | 2.42 | 2.38 |
| Group 6 | 2.30 | 2.42 | 2.36 |

Sampling was performed at predose, 2, 5, 15, and 30 minutes, 1, 2, 4, and 8 hours post dose. Blood samples were collected, placed into chilled tubes containing sodium heparin, and kept chilled until centrifuged at 4° C. at 13,000 rpm for 5 minutes. Plasma was separated and stored at −60° C. to −80° C. until analyzed.

Plasma levels of the compounds were determined by LC-MS/MS. Quantification of the compounds in plasma was performed against a calibration curve generated by serial dilution of a test article DMSO stock solution into blank heparinized rat plasma (50, 100, 200, 500, 1,000, 2,000, 5,000, and 10,000 ng/mL final concentrations of free drug). Quality control samples were prepared in the same fashion (250, 1,000, and 5,000 ng/mL final concentrations of free drug). The standard curve and quality control DMSO stock solutions were derived from independent weightings.

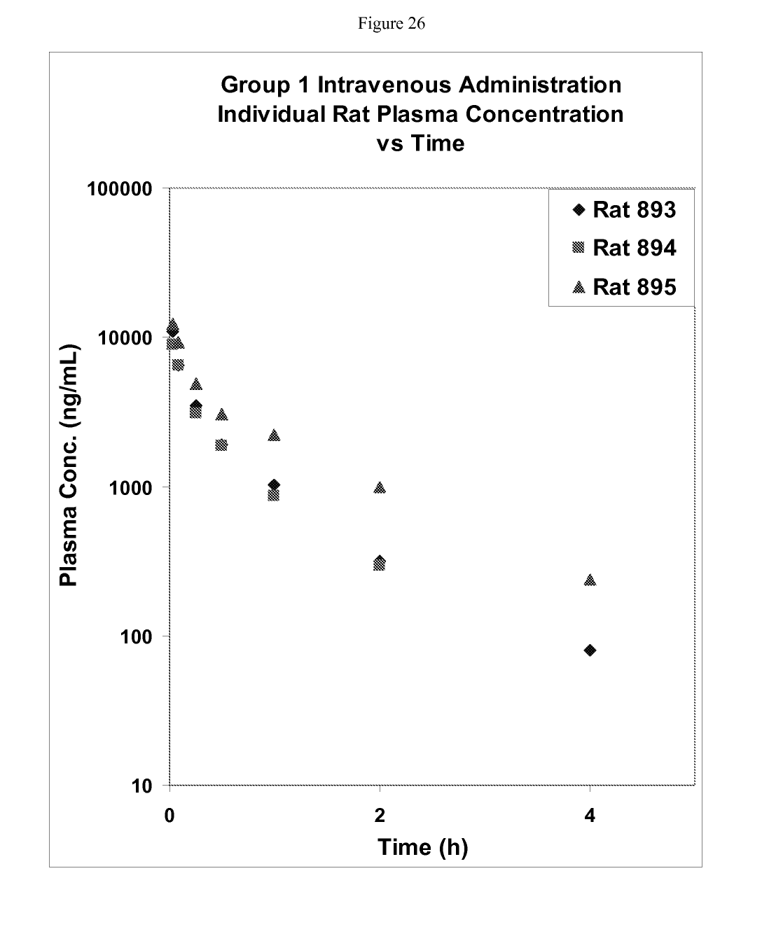

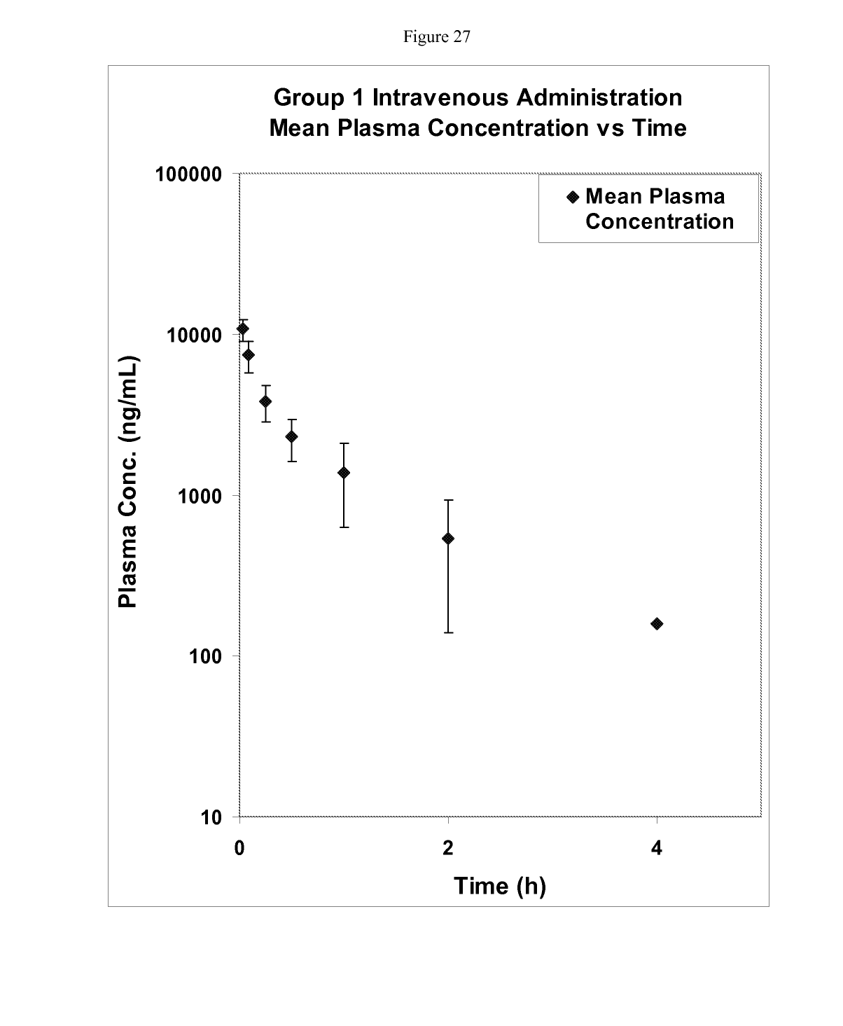

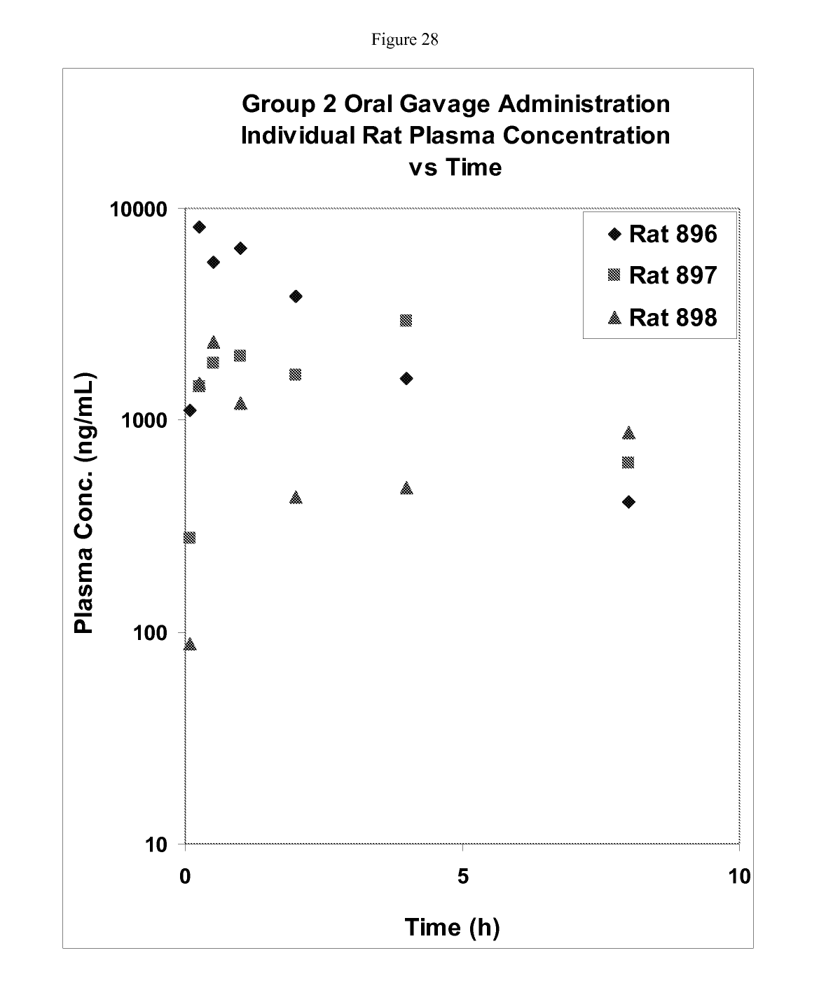

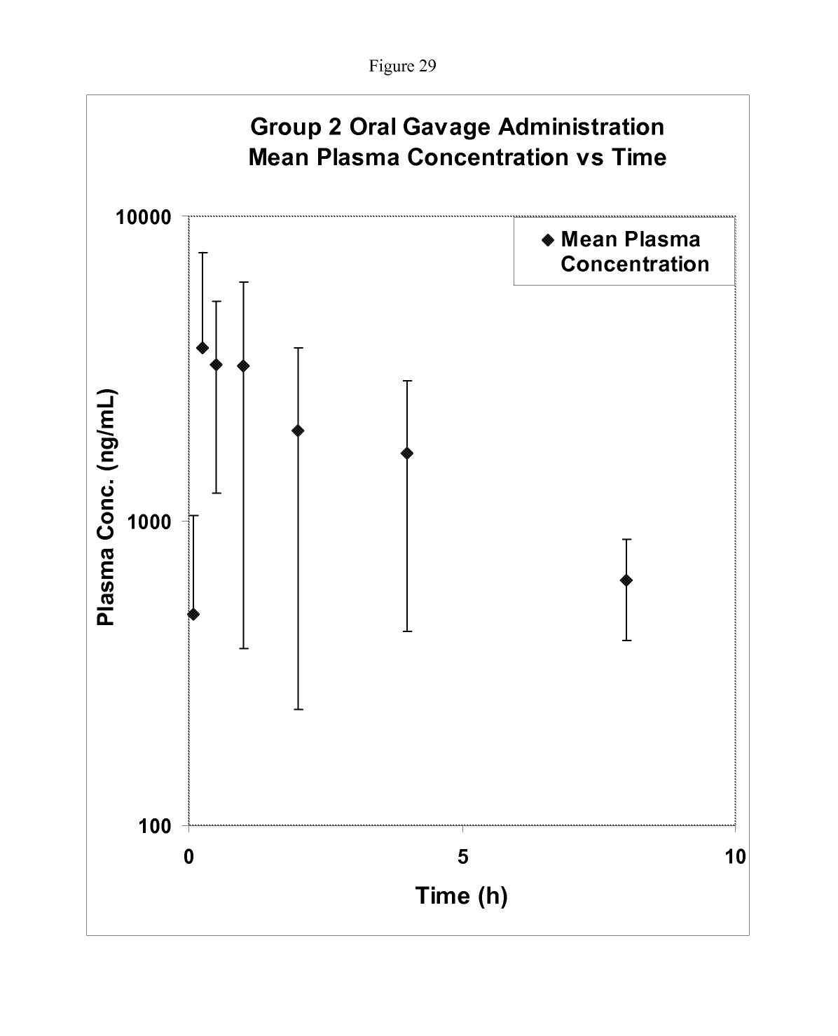

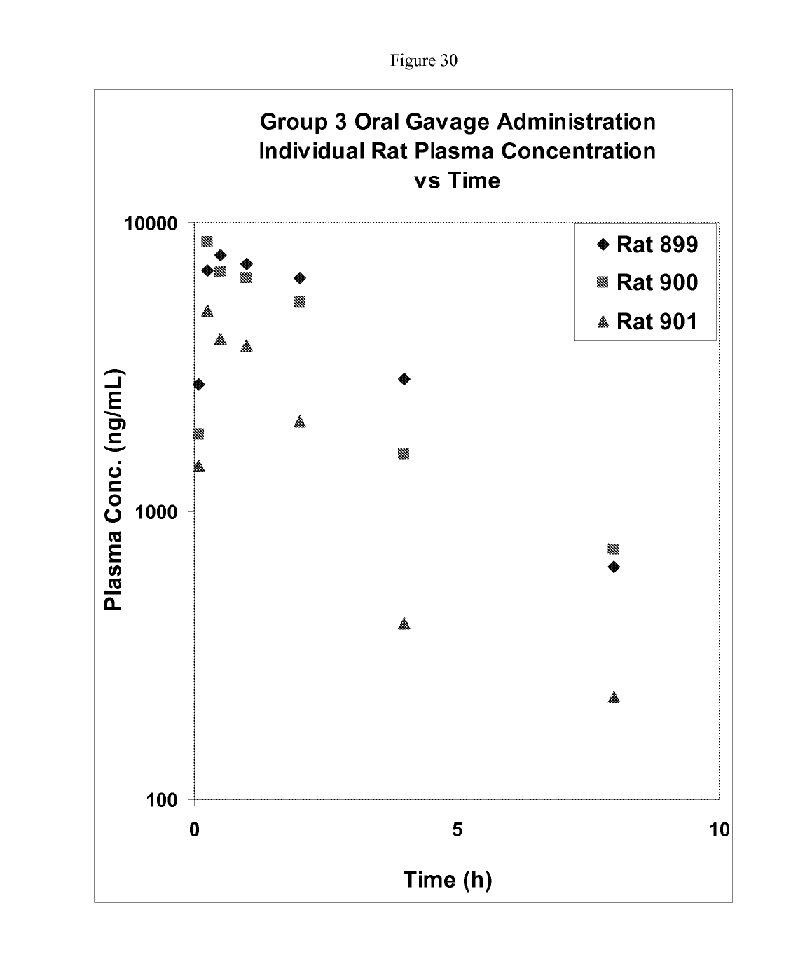

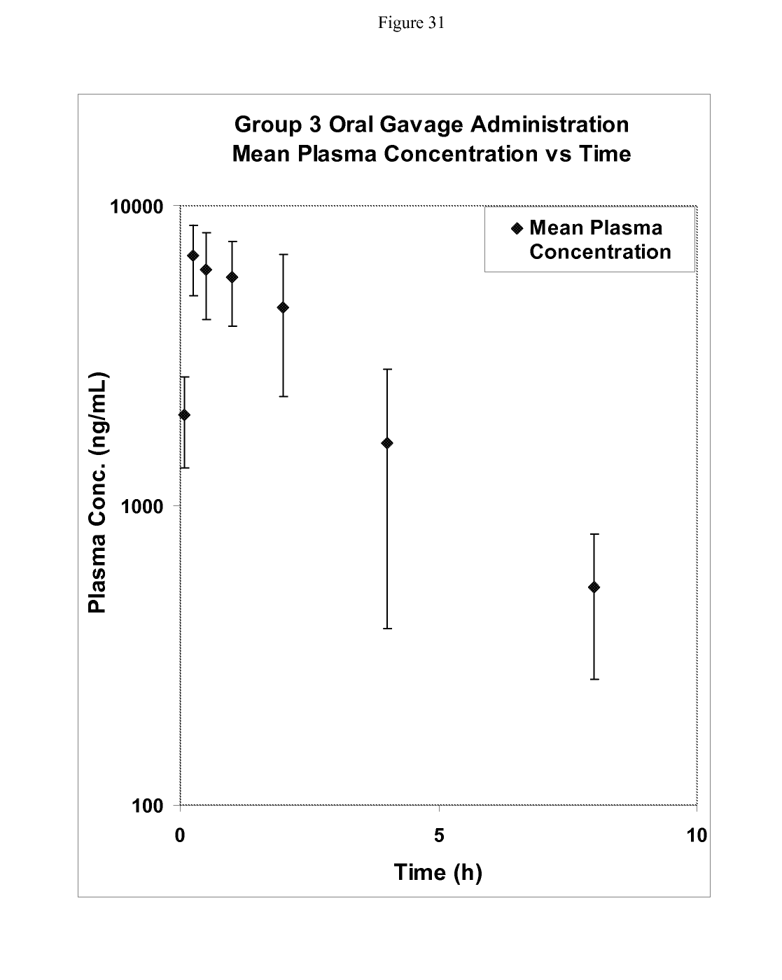

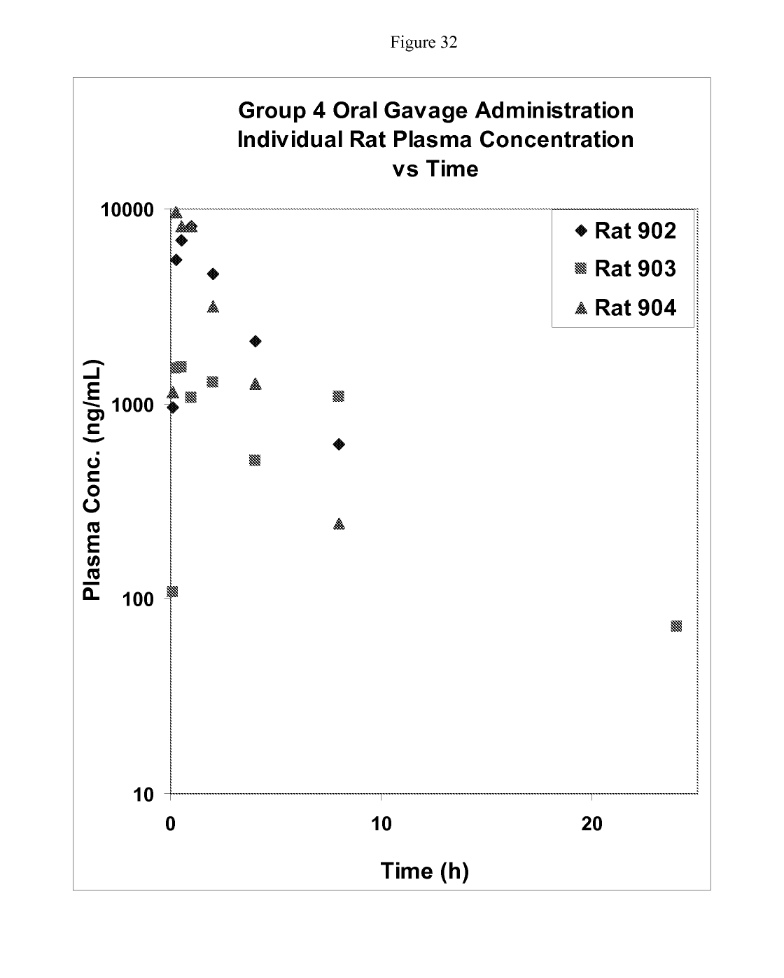

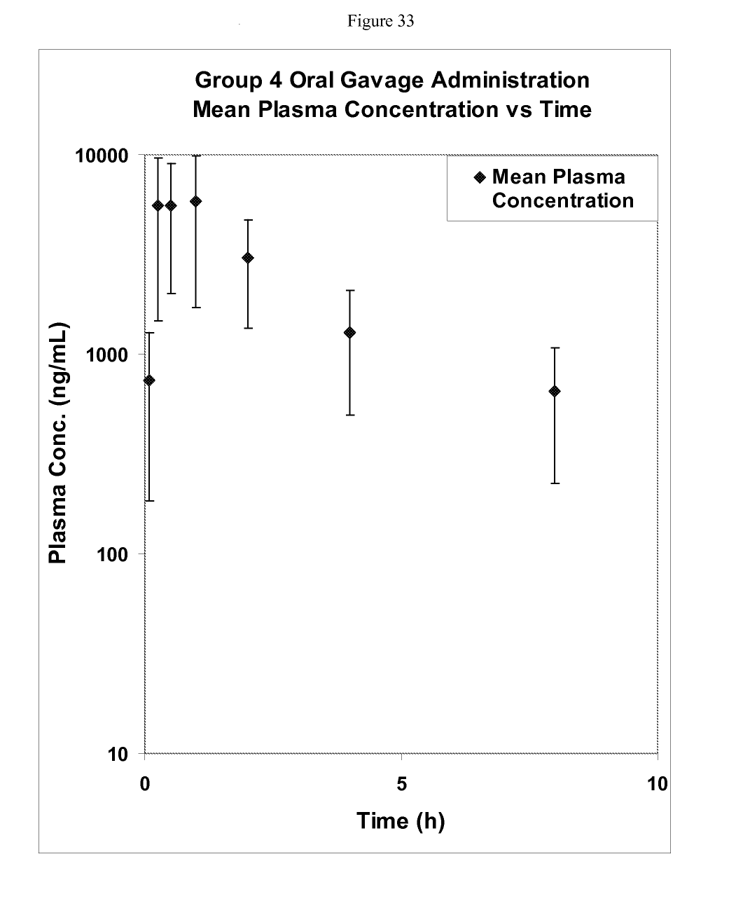

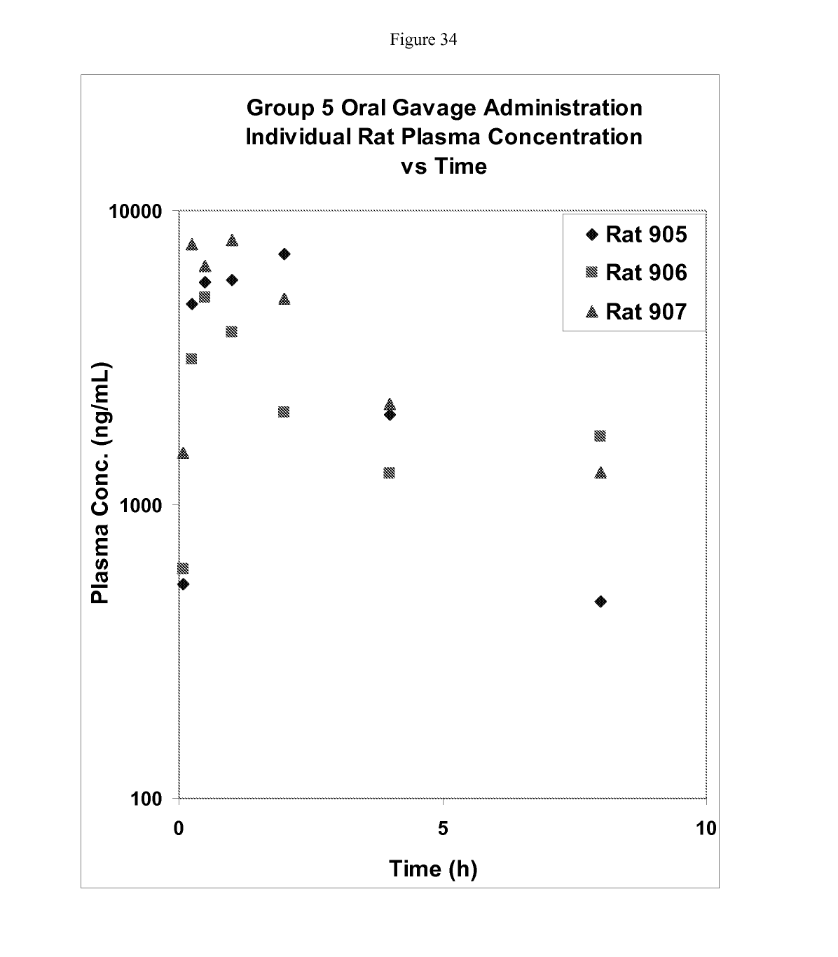

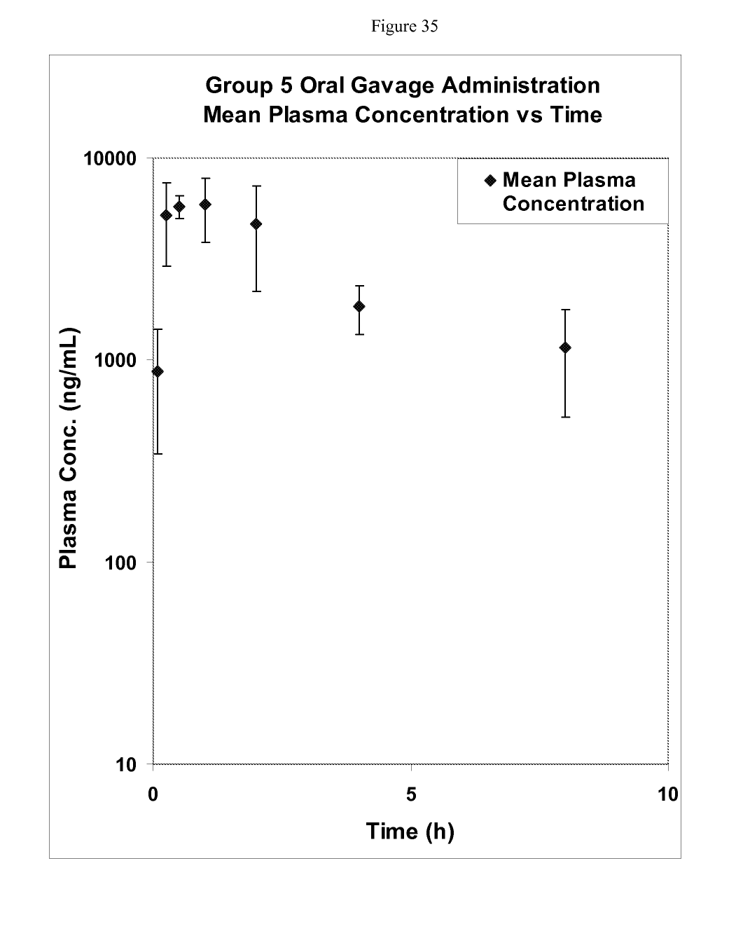

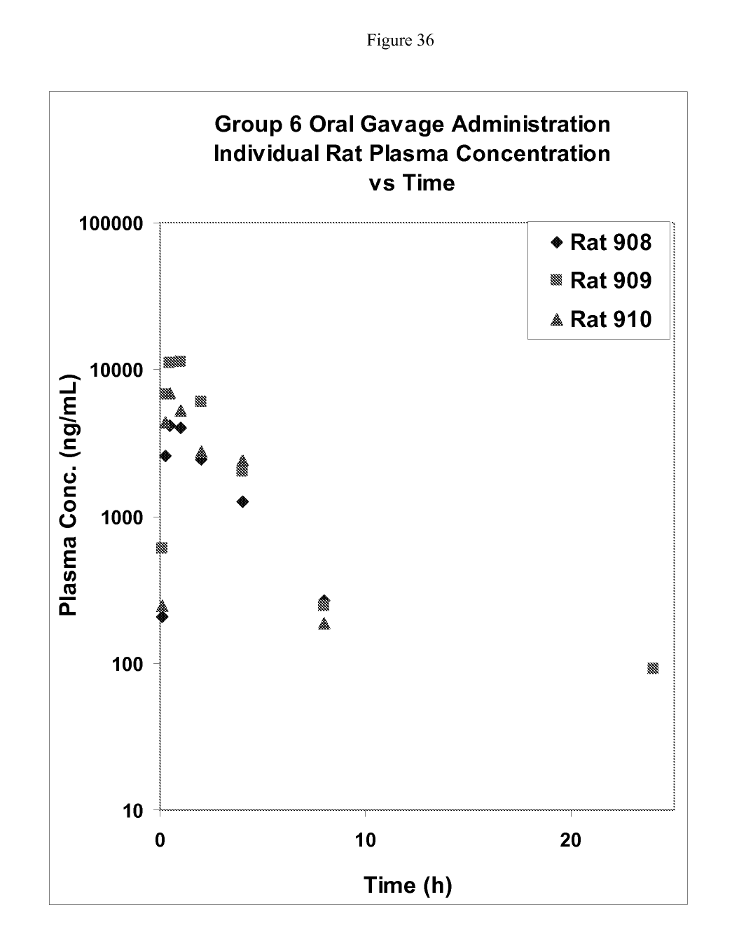

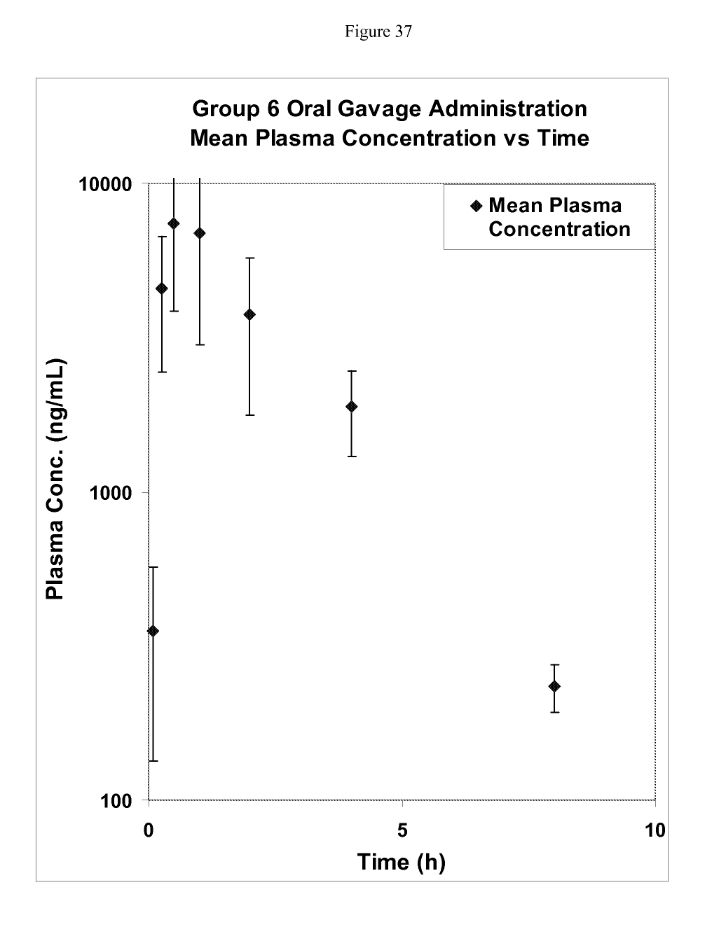

Plasma concentrations of tested compounds are shown in Tables 4, 7, 10, 13, and 16. Plasma concentrations versus time data are plotted in FIGS. 26 through 37.

Pharmacokinetic parameters of tested compounds are shown in Tables 5, 8, 11, 14, and 17.

All data are expressed as ng/mL of the free drug. Samples that were below the limit of quantification (“bloq”) were assigned a value of zero for the mean concentration calculations.

### General Scheme for Di-Salt Synthesis

Generally, DSB di-salts of the present invention can be prepared by reacting DSB in its free acid form with a suitable organic or inorganic base to produce a salt, and optionally isolating the salt. Di-salts of the present invention are made by mixing 2 or more equivalents of a basic or cation-forming compound, such as (+)-arginine, choline hydroxide, diethylamine, or diethanolamine, an aqueous solution, and 1 equivalent of DSB in an ethanol solution, and, optionally, isolating the DSB di-salt as a solid from the resultant solution. The mixing can occur in the presence of a cyclodextrin, such as hydroxypropyl-β-cyclodextrin. These salts can be prepared in situ during the final isolation and purification of the compounds or by separately reacting the purified compound in its free acid form with a suitable base and isolating the salt thus formed.

### General Scheme for Polymorph Synthesis

Many processes of the present invention involve crystallization out of a particular solvent. One skilled in the art would appreciate that the conditions concerning crystallization can be modified without affecting the form of the polymorph obtained. For example, when mixing a DSB di-salt in a suitable solvent to form a solution, warming of the mixture can be necessary to completely dissolve the starting material. If warming does not clarify the mixture, the mixture can be diluted or filtered. To filter, the hot mixture can be passed through paper, glass fiber or other membrane material, or a clarifying agent such as Celite (diatomaceous earth). Depending upon the equipment used and the concentration and temperature of the solution, the filtration apparatus may need to be preheated to avoid premature crystallization.

The conditions can also be changed to induce precipitation. One technique useful in inducing precipitation is to reduce the solubility of a DSB di-salt in the solvent. The solubility of the solvent can be reduced, for example, by cooling the solvent. Another technique useful in inducing precipitation is to introduce a seed crystal of the desired polymorph into the solution.

In one embodiment, a second solvent is added to a solution to decrease its solubility for a particular compound, thus resulting in precipitation. In another embodiment, a second solvent is added to an oily residue or a gummy material, wherein the low solubility of the second solvent for a particular compound results in precipitation of that compound.

In one embodiment, crystallization is accelerated by seeding with a crystal of the product or scratching the inner surface of the crystallization vessel with a glass rod. In another embodiment, crystallization can occur spontaneously without any inducement. All that is necessary to be within the scope of the claims relating to processes of producing a polymorph of a DSB di-salt is to form a precipitate or crystal.

Those of ordinary skill in the art will appreciate that several solvent evaporation or solution saturation techniques are useful in practicing the present invention including without limitation: introducing a shear flow; introducing a heated element such as heat transfer plates, IR lamps, microwave systems; distillation with an optional sheer flow wherein the distillation can be preformed at atmospheric pressure or under vacuum; static evaporation; reducing the temperature of the DSB di-salt solution; and, thin film evaporation techniques such as rotary evaporation, spin-off evaporation, rising and falling film evaporation, submerged evaporation, and wiped film evaporation.

Each solid was homogenized and characterized for crystallinity by PXRD according to Method G, and for thermal properties by DSC and TGA according to Methods C and D respectively. Recrystallization from methanol or ethanol also provided DSB-2 choline Form I-c.

### Example 1

### Preparation and Characterization of DSB Free Acid

The free acid of DSB is prepared by the processes disclosed in U.S. Pat. No. 5,679,828 and U.S. application Ser. No. 11/081,802.

Microscopic observations included columnar-shaped birefringent crystals having an average size of about 20 microns wide by about 350 microns long.

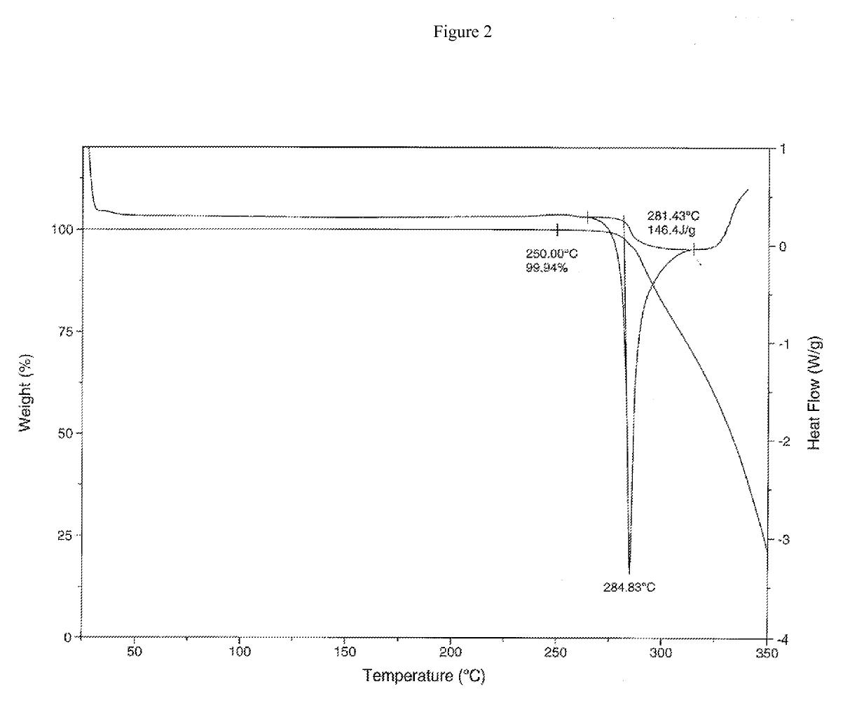

Differential scanning calorimetry-thermogravimetric analysis (DSC/TGA), performed in accordance with Method C and as depicted in FIG. 2, exhibited one endotherm occurring at about 280° C. which is attributed to melting.

The free acid contains two ionization sites as carboxylic acid moieties capable of forming salts in the pH range of interest: calculated pKₐ values were 4.1 and 5.3.

Hot stage microscopy performed according to Method E indicated that the sample melted at about 270° C.

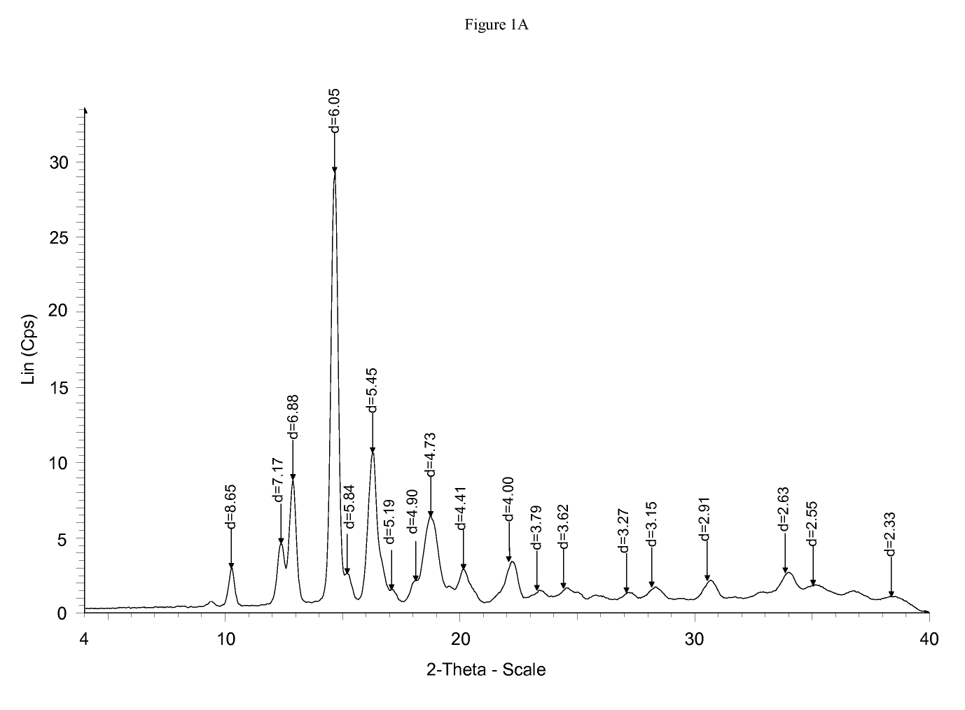

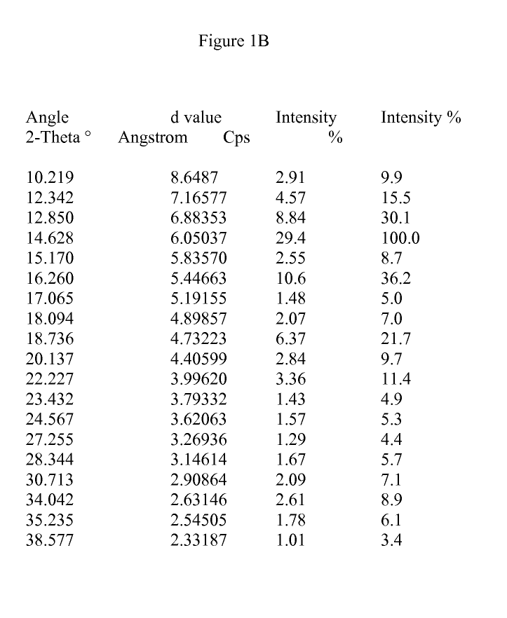

As shown in the PXRD, performed in accordance with Method G and depicted in FIG. 1, the DSB free acid is crystalline.

The solubility profile of the prepared DSB free acid, performed in accordance with Method B, is shown in Table 2.

TABLE 2
Solubility Profile of the DSB Free Acid

| | DSB Free |
|---|---|
| | Acid |
| Solvent | (μg/mL) |
| Methanol | 7.5 |
| Ethanol | 15 |
| 2,2,2- | 2.5 |
| Trifluoroethanol | |
| 1-Propanol | 6 |
| 2-Propanol | 6 |
| Water | 0.35 |

### Example 2

### Preparation and Characterization of DSB (+)-Arginine Disalt

One embodiment of the present invention comprises an (+)-arginine salt of DSB. In one embodiment the (+)-arginine salt of DSB is the bis-(+)-arginine salt of DSB (“DSB-2 (+)-arginine”). The bis-(+)-arginine salt of DSB has about two (+)-arginine molecules per DSB molecule; has a molecular formula of about C₃₆H₅₆O₆.[C₆H₁₄N₄O₂]₂, a molecular weight of about 933.23 and has the following structural formula:

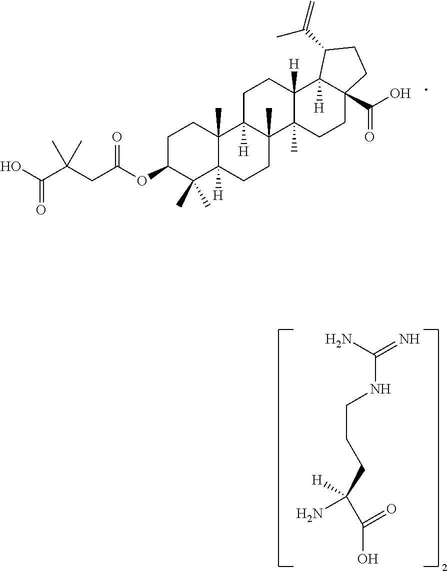

In one embodiment the DSB bis-(+)-arginine salt is DSB-2 (+)-arginine Form IA. DSB-2 (+)-arginine Form I was prepared by dissolving DSB free acid in ethanol. The arginine was dissolved in water. Two equivalents of the (+)-arginine solution were mixed with one equivalent of the DSB solution to form the bis-(+)-arginine salt of DSB. A clear solution resulted with no precipitation. The solution was dried slowly using a TurboVap workstation at 25° C. and 5 psi nitrogen shear followed by drying in a vacuum oven at 35° C.

The solubility profile of the DSB-2 (+)-arginine Form I-a, performed in accordance with Method B, is shown in Table 3.

TABLE 3
Solubility Profile of DSB-2 (+)-arginine Form I-a Compared to Free Acid

| | (+)-Arginine | |
|---|---|---|
| | Disalt Form | Free |
| | IA | Acid |
| Solvent | (μg/mL) | (μg/mL) |
| Methanol | 1.9 | 7.5 |
| Ethanol | 0.9 | 15 |
| 2,2,2-Trifluoroethanol | 0.9 | 2.5 |
| 1-Propanol | 0.6 | 6.0 |
| 2-Propanol | <0.5 | 6.0 |
| Water | <0.5 | <0.5 |

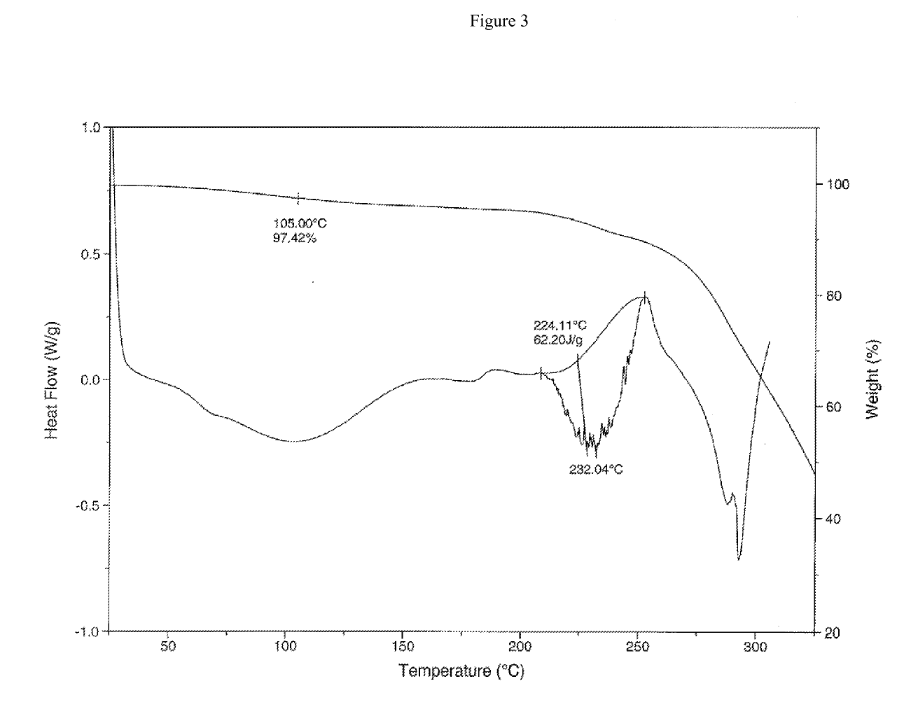

DSC of DSB-2 (+)-arginine Form I-a, performed in accordance with Method C and as depicted in FIG. 3, exhibited two endotherms: the first endotherm occurring at about 92° C. to about 99° C. is attributed to melting; the second endotherm starting about 260° C. was observed to be noisy and is attributed to decomposition. TGA of DSB-2 (+)-arginine Form I-a, performed in accordance with Method D and as depicted in FIG. 3, demonstrated a mass loss of about 4 wt % at about 100° C.

Hot stage microscopy of DSB-2 (+)-arginine Form I-a, performed according to Method E indicated that the sample melted over a range of about 92° C. to about 99° C.

Microscopic observations included a mixture of crystalline plates, plate fragments, and other areas that did not appear to be crystalline. The sample lacked consistent birefringence.

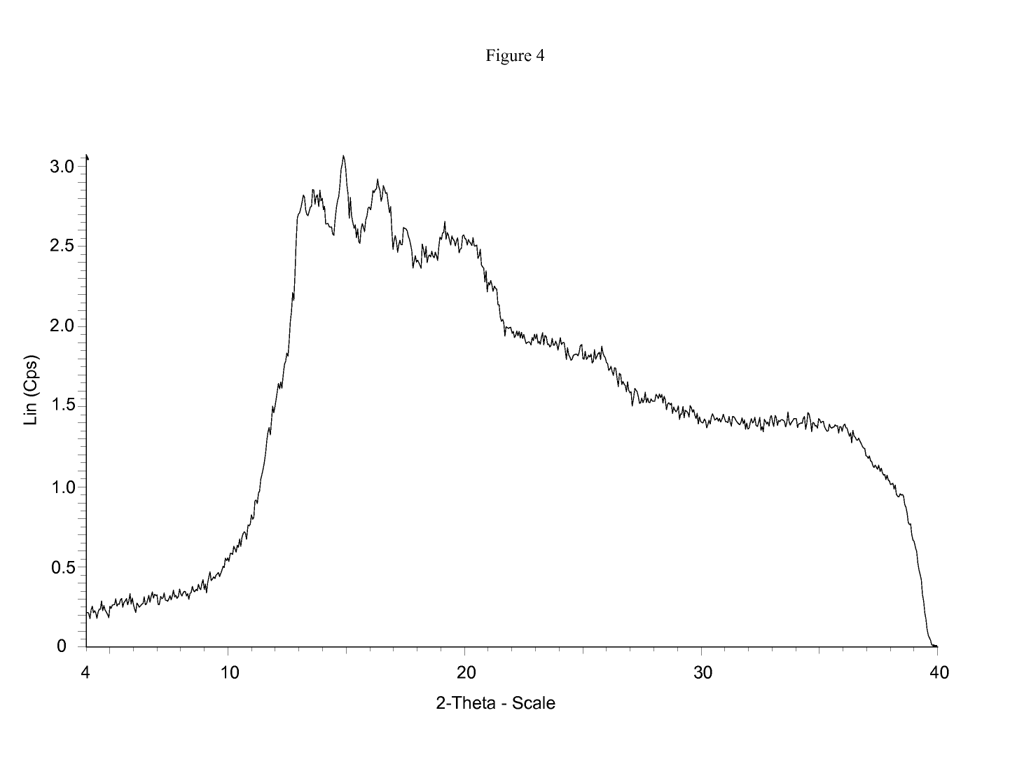

As shown in the PXRD of DSB (+)-arginine disalt Form I-a, performed in accordance with Method G and depicted in FIG. 4, the DSB (+)-arginine disalt Form IA is amorphous.

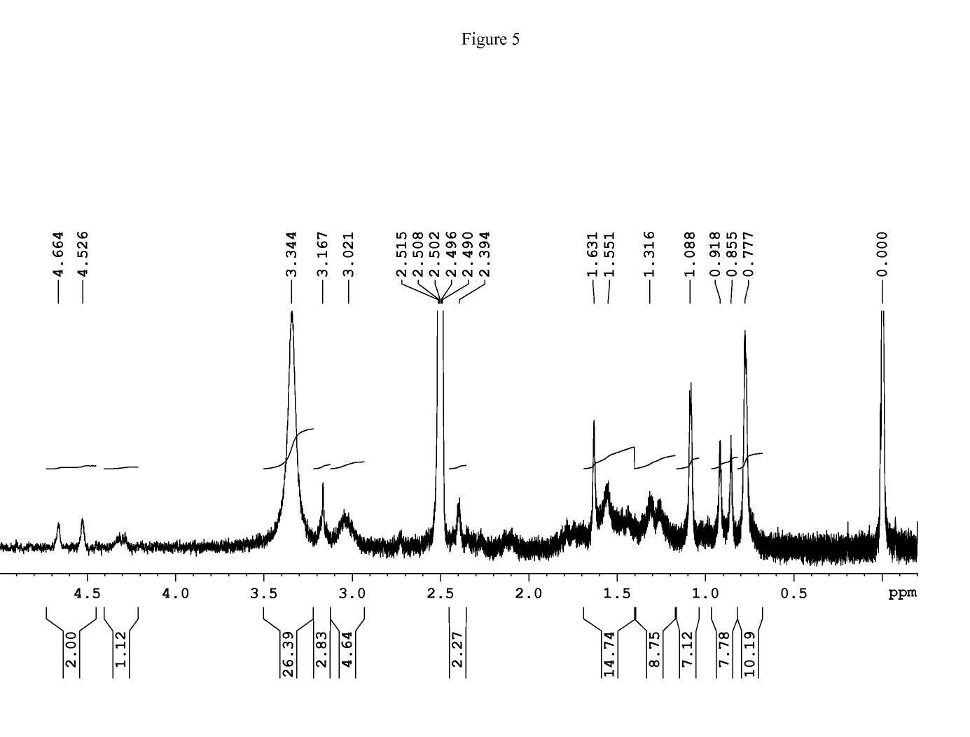

As shown in FIG. 5, ¹H FT-NMR performed in accordance with Method A confirmed that the stoichiometric ratio of the DSB free acid to (+)-arginine was 1:2. Chemical shift values for the distinguishing peaks appear at about 4.664, 4.526, 3.344, 3.167, 3.021, 2.515, 2.508, 2.502, 2.496, 2.490, 2.394, 1.631, 1.551, 1.316, 1.088, 0.918, 0.855, 0.777, 0.023, 0.018, 0.010, 0.008, 0.005, 0.000, −0.005, −0.011, and −0.032 ppm.

In one embodiment the DSB di-(+)-arginine salt is DSB-2 (+)-arginine Form II-a. DSB-2 (+)-arginine Form II-a, was prepared by dissolving DSB-2 (+)-arginine Form I-a, in 2,2,2-trifluoroethanol and recrystallizing the resultant solution. Recrystallization is achieved by dissolving the DSB-2 (+)-arginine Form I-a, in a minimum volume of 2,2,2-trifluoroethanol to create a mixture, warming the mixture to create a warmed solution, removing any insoluble impurities by filtration, slowly cooling the warmed solution to crystallize DSB-2 (+)-arginine Form II-a, and filtering off the remaining liquid to isolate the recrystallized DSB-2 (+)-arginine Form II-a.

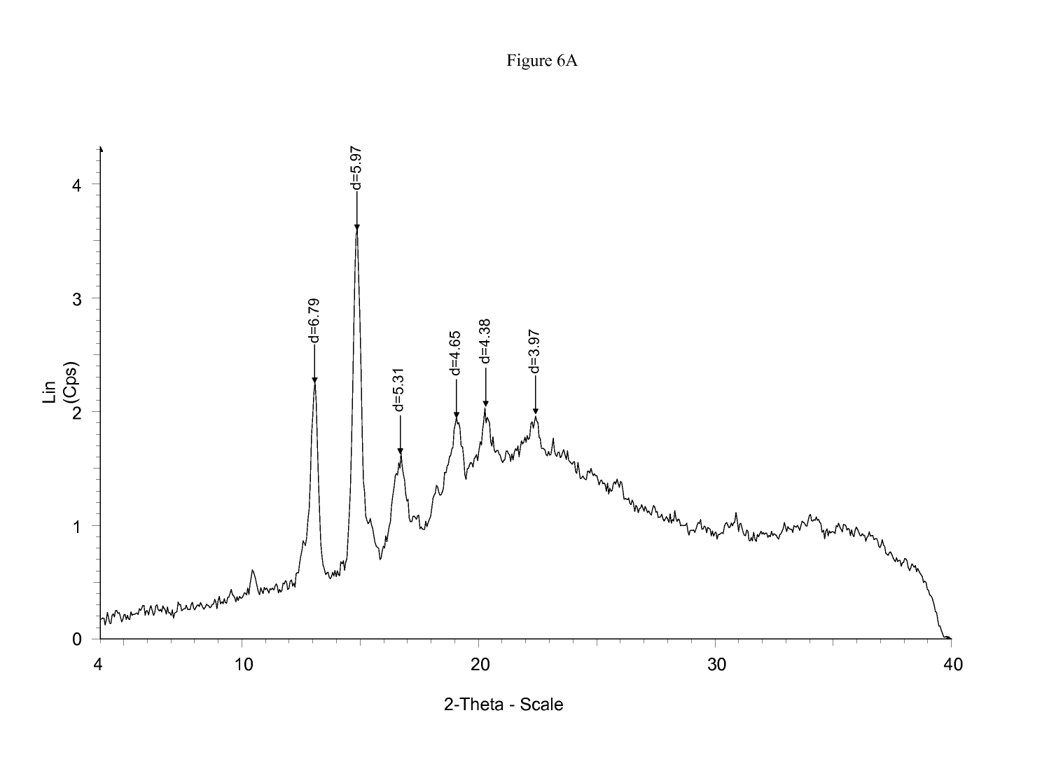

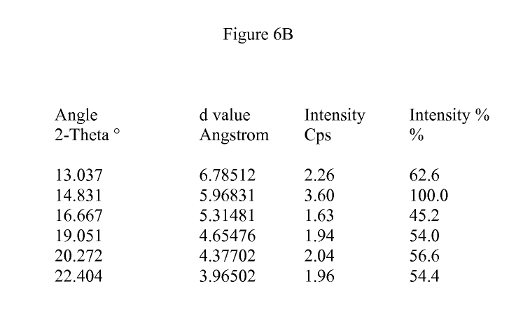

As shown in the PXRD of DSB-2 (+)-arginine Form II-a, performed in accordance with Method G and depicted in FIG. 6, the DSB (+)-arginine disalt is crystalline.

In some embodiments, DSB-2 (+)-arginine Form II-a, is characterized by an X-ray powder diffraction pattern exhibiting at least one diffraction peak corresponding to d-spacings of about 6.79, 5.97, 5.31, 4.65, 4.38, or 3.97 angstroms⁻¹.

In some embodiments, DSB-2 (+)-arginine Form II-a, is characterized by the X-ray powder diffraction pattern of FIG. 6.

As shown in Tables 4 and 5, in vivo pharmacokinetic studies performed in accordance with Method H demonstrate the surprising oral bioavailability of the (+)-arginine disalt of DSB (34.7% greater than the NMG salt). Such a marked increase in bioavailability makes formulation of an (+)-arginine disalt of DSB feasible as an oral dosage form. Requiring the incorporation of less active ingredient would result in proportionally less excipient providing the benefits of (i) reducing the overall mass and size of an oral dosage form; (ii) increasing the amount of active ingredient relative to a solid dosage form comprising a less bioavailable DSB salt; or (iii) a combination of (i) and (ii).

TABLE 4
Comparative Mean Plasma Concentrations (ng/mL) of
DSB-2 NMG & DSB-2 (+)-Arginine Following Administration
by Oral Gavage to Group 2 Rats

| | Time (h) | | | | | | |
|---|---|---|---|---|---|---|---|
| | 0.083 | 0.25 | 0.5 | 1 | 2 | 4 | 8 |
| Plasma | 491.0 | 3706.7 | 3246.7 | 3236.7 | 1973.7 | 1662.3 | 635.7 |
| Concen- | | | | | | | |
| tration | | | | | | | |
| (DSB-2 | | | | | | | |
| NMG) | | | | | | | |
| Plasma | 2003.3 | 6796.7 | 6140.0 | 5793.3 | 4590.0 | 1615.3 | 532.3 |
| Concen- | | | | | | | |
| tration | | | | | | | |
| DSB-2 | | | | | | | |
| (+)-Ar- | | | | | | | |
| ginine | | | | | | | |

TABLE 5
Comparative Mean Pharmacokinetic Parameters of DSB-2 NMG &
DSB-2 (+)-Arginine Following Administration to Group 2 Rats

| | AUC_{24 h} | | | % |
|---|---|---|---|---|
| Group-Route | (h * ng/mL) | Cₘₐₓ (ng/mL) | Tₘₐₓ (h) | Bioavailability |
| DSB-2 NMG | 6890.6 | 13785.9 | 0.0 | 100.0 |
| Intravenous | | | | |
| DSB-2 NMG | 18041.1 | 4480.0 | 1.6 | 26.2 |
| Oral Gavage | | | | |
| DSB-2 (+)- | 24342.9 | 7093.3 | 0.33 | 35.3 |
| Arginine Oral | | | | |
| Gavage | | | | |

In some embodiments of the present invention, the DSB-2 (+)-arginine drug substance consists of substantially phase pure DSB-2 (+)-arginine Form I-a.

In some embodiments of the present invention, the DSB-2 (+)-arginine drug substance comprises at least about 90% DSB-2 (+)-arginine Form I-a, relative to all other solid state forms of DSB-2 (+)-arginine present in the DSB-2 (+)-arginine drug substance.

In some embodiments of the present invention, the DSB-2 (+)-arginine drug substance comprises at least about 75% DSB-2 (+)-arginine Form I-a, relative to all other solid state forms of DSB-2 (+)-arginine present in the DSB-2 (+)-arginine drug substance.

In some embodiments of the present invention, the DSB-2 (+)-arginine drug substance comprises at least a detectable amount of DSB-2 (+)-arginine Form I-a, relative to all other solid state forms of DSB-2 (+)-arginine present in the DSB-2 (+)-arginine drug substance.

In some embodiments of the present invention, the DSB-2 (+)-arginine drug substance consists of substantially phase pure DSB-2 (+)-arginine Form II-a.

In some embodiments of the present invention, the DSB-2 (+)-arginine drug substance comprises at least about 90% DSB-2 (+)-arginine Form II-a, relative to all other solid state forms of DSB-2 (+)-arginine present in the DSB-2 (+)-arginine drug substance.

In some embodiments of the present invention, the DSB-2 (+)-arginine drug substance comprises at least about DSB-2 (+)-arginine Form II-a, relative to all other solid state forms of DSB-2 (+)-arginine present in the DSB-2 (+)-arginine drug substance.

In some embodiments of the present invention, the DSB-2 (+)-arginine drug substance comprises at least a detectable amount of DSB-2 (+)-arginine Form II-a, relative to all other solid state forms of DSB-2 (+)-arginine present in the DSB-2 (+)-arginine drug substance.

One embodiment of the present invention comprises a pharmaceutical composition comprising an (+)-arginine salt of DSB, such as the bis-(+)-arginine salt of DSB, and a pharmaceutically acceptable excipient.

One embodiment of the present invention comprises a method of using a pharmaceutical composition that comprises an (+)-arginine salt of DSB, such as the bis-(+)-arginine salt of DSB for treating, in a human subject, a retroviral infection, such as HIV.

### Example 3

### Preparation and Characterization of DSB Choline Disalt

One embodiment of the present invention comprises a choline salt of DSB. In one embodiment the choline salt of DSB is the bis-choline salt of DSB. The bis-choline salt of DSB has about two choline molecules per DSB molecule; has a molecular formula of about C₃₆H₅₄O₆.[C₅H₁₄NO]₂, a molecular weight of about 791.15 and has the following formula:

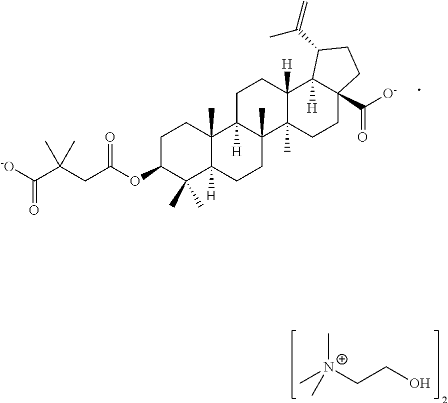

In one embodiment the DSB bis-choline salt is DSB-2 choline Form I-c. DSB-2 choline Form I-c was prepared by dissolving DSB free acid in ethanol. Choline hydroxide was dissolved in water. Two equivalents of the choline hydroxide solution were mixed with one equivalent of the DSB solution to form the di-choline salt of DSB. A clear solution resulted with no precipitation. The solution was dried slowly using a TurboVap workstation at 25° C. and 5 psi nitrogen shear followed by drying in a vacuum oven at 35° C. Recrystallization from methanol or ethanol also provided DSB-2 choline Form I-c.

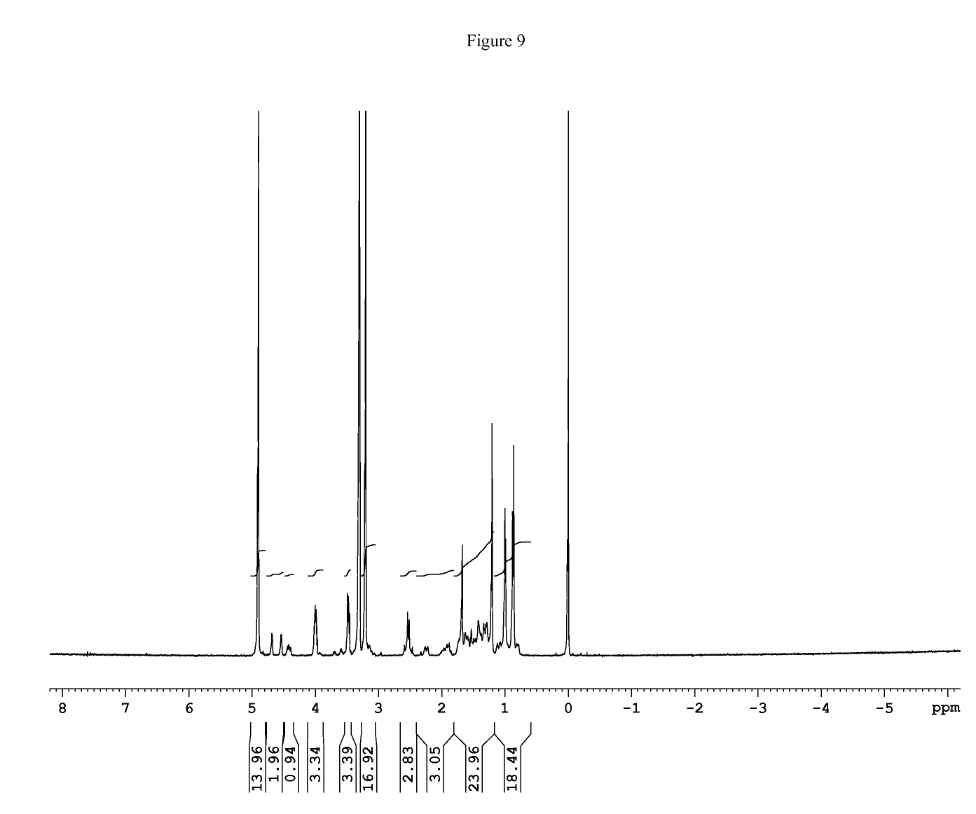

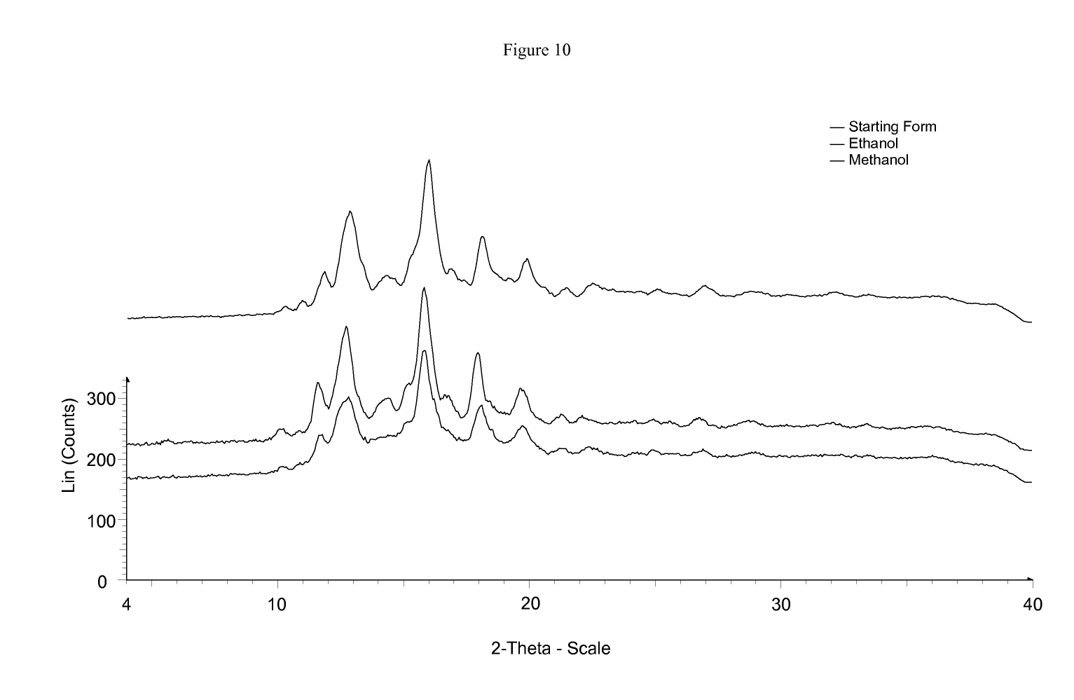

As shown in FIG. 9, ¹H FT-NMR performed in accordance with Method A confirmed that the stoichiometric ratio of the DSB free acid to choline was 1:2. Chemical shift values for the distinguishing peaks appear at about 5.92, 4.72, 4.59, 4.40, 4.03, 3.55, 3.37, 3.23, 2.56, 1.70, 1.21, 1.03, 0.88, and 0.00 ppm.

The solubility profile of the DSB choline disalt, performed in accordance with Method B, is shown in Table 6.

TABLE 6
Solubility profile of the DSB Choline disalt Compared to Free Acid

| | Choline | Free |
|---|---|---|
| | Disalt | Acid |
| Solvent | (μg/mL) | (μg/mL) |
| Methanol | >40 | 7.5 |
| Ethanol | >40 | 15 |
| 2,2,2- | >40 | 2.5 |
| Trifluoroethanol | | |
| 1-Propanol | >40 | 6.0 |
| 2-Propanol | 20 | 6.0 |
| Water | 8 | <0.5 |

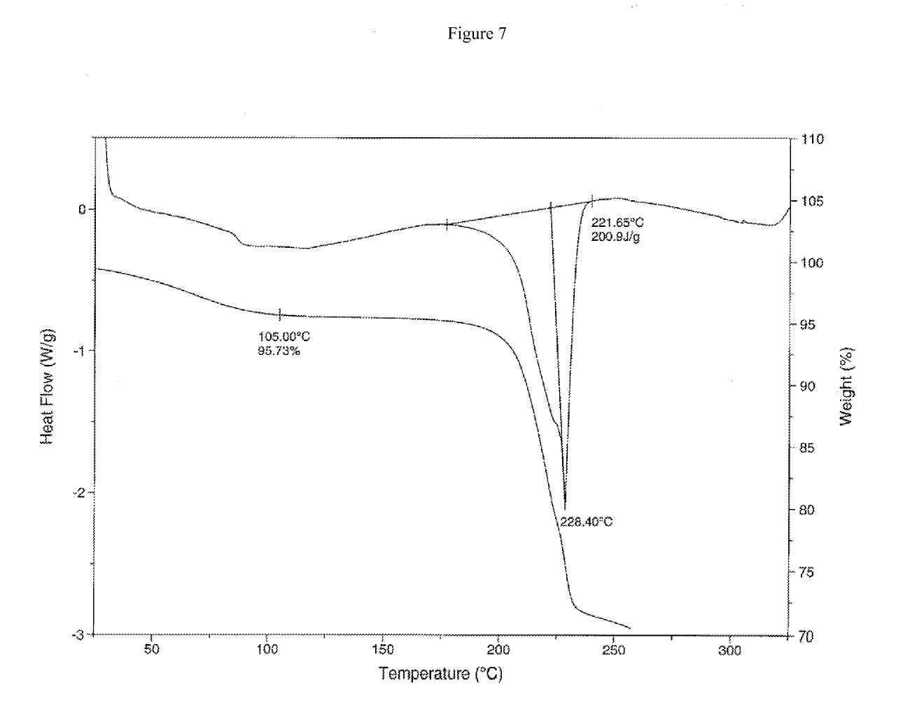

DSC, performed in accordance with Method C and as depicted in FIG. 7, exhibited an endotherm occurring at about 205° C. to about 225° C. and is attributed to melting. TGA, performed in accordance with Method D and as depicted in FIG. 7, demonstrated a mass loss of about 4 wt % at about 105° C.

Hot stage microscopy according to Method E indicated that the sample melted over a range of about 205° C. to about 225° C.

Microscopic observations included a mixture of crystalline plates, plate fragments, and other areas that did not appear to be crystalline.

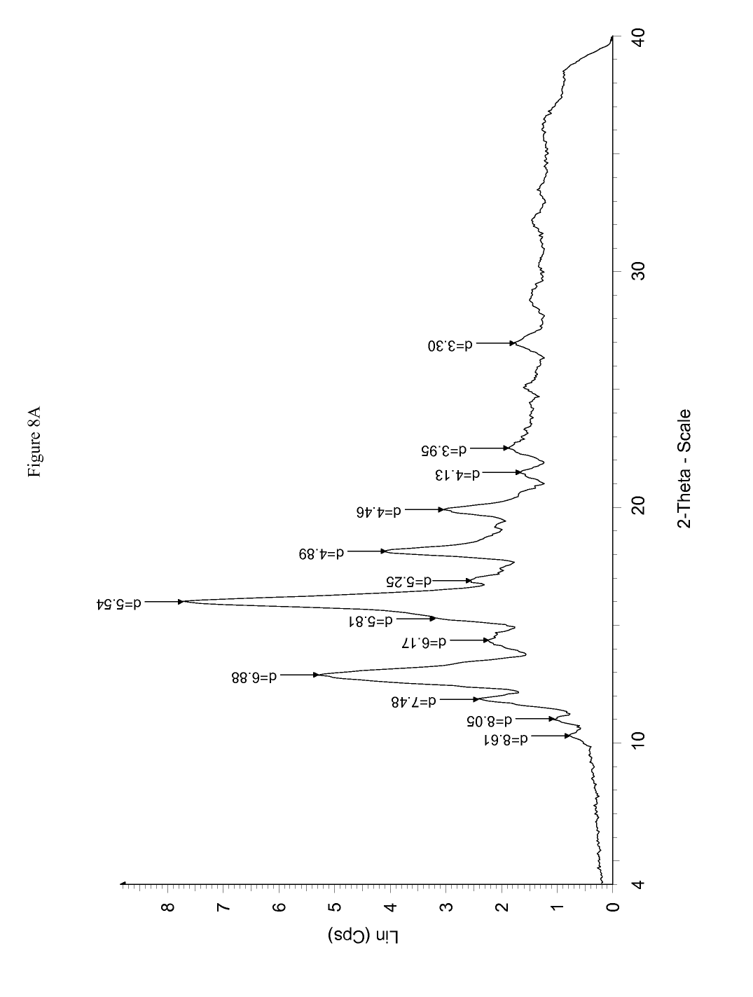

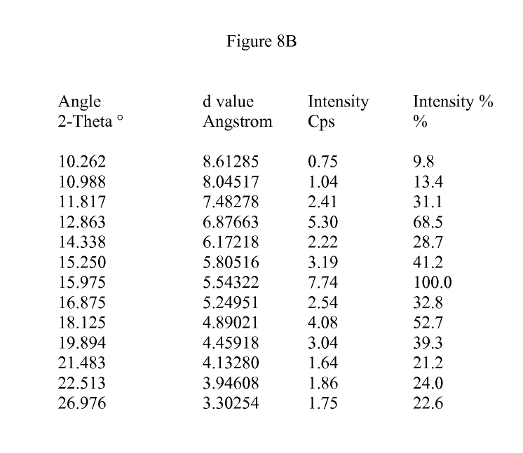

As shown in PXRD of FIG. 8 performed in accordance with Method G, the DSB choline disalt was a crystalline compound.

In some embodiments, DSB-2 choline Form I-c is characterized by the X-ray powder diffraction pattern of FIG. 8.

In some embodiments, DSB-2 choline Form I-c is characterized by an X-ray powder diffraction pattern exhibiting at least one diffraction peak corresponding to d-spacings of about 8.61, 8.05, 7.48, 6.88, 6.17, 5.80, 5.54, 5.25, 4.89, 4.46, 4.13, 3.95, or 3.30 angstroms⁻¹.

In one embodiment the DSB di-choline salt is DSB-2 choline Form II-c.

DSB-2 choline Form II-c was prepared by dissolving DSB-2 choline Form I-c in 1-propanol or 2-propanol and recrystallizing the resultant solution, and, optionally, filtering off the remaining liquid to isolate the recrystallized DSB-2 choline Form II-c.

In one embodiment the DSB di-choline salt is DSB-2 choline Form III-c. DSB-2 choline Form III-c was prepared by dissolving DSB-2 choline Form I-c in N,N-dimethylformamide or N,N-dimethylacetamide and recrystallizing the resultant solution, and, optionally, filtering off the remaining liquid to isolate the recrystallized DSB-2 choline Form III-c.

In one embodiment the DSB bis-choline salt is DSB-2 choline Form IV-c. DSB-2 choline Form IV-c was prepared by dissolving the DSB-2 choline Form I-c in a minimum volume of acetonitrile to create a solution, warming the solution, removing any insoluble impurities by filtration, and slowly cooling the warmed solution to provide amorphous DSB-2 choline Form IV-c. The amorphous nature of DSB-2 choline Form IV-c is demonstrated by failing to exhibit a long range crystal order. Recrystallization from water provided a disalt also failing to exhibit a long range crystal order.

As shown in Tables 7 and 8, in vivo pharmacokinetic studies performed in accordance with Method H demonstrate the dramatically increased solubility of the choline DSB disalt relative to the free acid. Such marked increases in solubility aid may enhance bioavailability and improve manufacturability of drug substances or drug products comprising the choline DSB disalt. The increase in solubility exhibited by the choline DSB disalt allows for flexibility in the formulation process, for example in the use of a choline disalt of DSB in intravenous administration, where increased water solubility is important. The greater than 16 fold increase in solubility as compared to the DSB free acid surprised experts, as the parent compound, DSB, is generally considered to be lipophilic, exhibiting very low solubility in aqueous systems.

TABLE 7
Comparative Mean Plasma Concentrations (ng/mL) of DSB-2 NMG &
DSB-2 Choline Following Administration by Oral Gavage to Group 3 Rats

| | Time (h) | | | | | | |
|---|---|---|---|---|---|---|---|
| | 0.083 | 0.25 | 0.5 | 1 | 2 | 4 | 8 |
| Plasma | 491.0 | 3706.7 | 3246.7 | 3236.7 | 1973.7 | 1662.3 | 635.7 |
| Concen- | | | | | | | |
| tration | | | | | | | |
| DSB-2 | | | | | | | |
| NMG | | | | | | | |
| Plasma | 738.0 | 5540.0 | 5530.0 | 5806.7 | 3030.0 | 1289.7 | 650.0 |
| Concen- | | | | | | | |
| tration | | | | | | | |
| DSB-2 | | | | | | | |
| Choline | | | | | | | |

TABLE 8
Comparative Mean Pharmacokinetic Parameters of DSB-2 NMG
& DSB-2 Choline Following Administration by Oral Gavage to
Group 3 Rats

| | AUC_{24 h} | | Tₘₐₓ | % |
|---|---|---|---|---|
| Group-Route | (h * ng/mL) | Cₘₐₓ (ng/mL) | (h) | Bioavailability |
| DSB-2 NMG | 6890.6 | 13785.9 | 0.0 | 100.0 |
| Intravenous | | | | |
| DSB-2 NMG | 18041.1 | 4480.0 | 1.6 | 26.2 |
| Oral Gavage | | | | |
| DSB-2 Choline | 20872.2 | 6460.0 | 0.58 | 30.3 |
| Oral Gavage | | | | |

In some embodiments of the present invention, the DSB-2 choline drug substance consists of substantially phase pure DSB-2 choline Form I-c.

In some embodiments of the present invention, the DSB-2 choline drug substance comprises at least about 90% DSB-2 choline Form I-c relative to all other solid state forms of DSB-2 choline present in the DSB-2 choline drug substance.

In some embodiments of the present invention, the DSB-2 choline drug substance comprises at least about 75% DSB-2 choline Form I-c relative to all other solid state forms of DSB-2 choline present in the DSB-2 choline drug substance.

In some embodiments of the present invention, the DSB-2 choline drug substance comprises at least a detectable amount of DSB-2 choline Form II-c relative to all other solid state forms of DSB-2 choline present in the DSB-2 choline drug substance.

In some embodiments of the present invention, the DSB-2 choline drug substance consists of substantially phase pure DSB-2 choline Form III-c.

In some embodiments of the present invention, the DSB-2 choline drug substance comprises at least about 90% DSB-2 choline Form II-c relative to all other solid state forms of DSB-2 choline present in the DSB-2 choline drug substance.

In some embodiments of the present invention, the DSB-2 choline drug substance comprises at least about 75% DSB-2 choline Form II-c relative to all other solid state forms of DSB-2 choline present in the DSB-2 choline drug substance.

In some embodiments of the present invention, the DSB-2 choline drug substance comprises at least a detectable amount of DSB-2 choline Form II-c relative to all other solid state forms of DSB-2 choline present in the DSB-2 choline drug substance.

In some embodiments of the present invention, the DSB-2 choline drug substance consists of substantially phase pure DSB-2 choline Form III-c.

In some embodiments of the present invention, the DSB-2 choline drug substance comprises at least about 90% DSB-2 choline Form III-c relative to all other solid state forms of DSB-2 choline present in the DSB-2 choline drug substance.

In some embodiments of the present invention, the DSB-2 choline drug substance comprises at least about 75% DSB-2 choline Form III-c relative to all other solid state forms of DSB-2 choline present in the DSB-2 choline drug substance.

In some embodiments of the present invention, the DSB-2 choline drug substance comprises at least a detectable amount of DSB-2 choline Form III-c relative to all other solid state forms of DSB-2 choline present in the DSB-2 choline drug substance.

In some embodiments of the present invention, the DSB-2 choline drug substance consists of substantially phase pure DSB-2 choline Form IV-c.

In some embodiments of the present invention, the DSB-2 choline drug substance comprises at least about 90% DSB-2 choline Form IV-c relative to all other solid state forms of DSB-2 choline present in the DSB-2 choline drug substance.

In some embodiments of the present invention, the DSB-2 choline drug substance comprises at least about 75% DSB-2 choline Form IV-c relative to all other solid state forms of DSB-2 choline present in the DSB-2 choline drug substance.

In some embodiments of the present invention, the DSB-2 choline drug substance comprises at least a detectable amount of DSB-2 choline Form IV-c relative to all other solid state forms of DSB-2 choline present in the DSB-2 choline drug substance.

One embodiment of the present invention comprises a pharmaceutical composition comprising a choline salt of DSB, such as the di-choline salt of DSB, and a pharmaceutically acceptable excipient.

One embodiment of the present invention comprises a method of preparing a choline salt of DSB. In one embodiment of the invention, the method of preparing the salt comprises mixing choline hydroxide and DSB in an aqueous ethanol solution to provide DSB 2 choline

Form I-c.

One embodiment of the present invention comprises a method of using a pharmaceutical composition that comprises an choline salt of DSB, such as the bis-choline salt of DSB for treating, in a human subject, a retroviral infection, such as HIV.

### Example 4

### Preparation and Characterization of DSB Diethanolamine Disalt

One embodiment of the present invention comprises a diethanolamine salt of DSB.

In one embodiment the diethanolamine salt of DSB is the diethanolamine disalt of DSB (“DSB-2 diethanolamine”). DSB-2 diethanolamine has about two diethanolamine molecules per DSB molecule; has a molecular formula of about C₃₆H₅₆O₆.[C₄H₁₁NO₂]₂, a molecular weight of about 795.1064 and has the following structural formula:

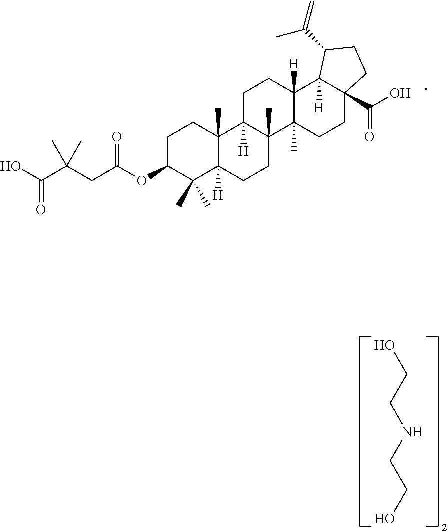

In one embodiment the DSB-2 diethanolamine is DSB-2 diethanolamine Form I-o. DSB-2 diethanolamine Form I-o was prepared by dissolving DSB free acid in methanol, ethanol, 1-propanol, 2-propanol, water, N,N-dimethylformamide, N,N-dimethylacetamide, acetone, ethyl acetate, or methylene chloride. Two equivalents of the diethanolamine solution were mixed with one equivalent of the DSB solution to form the diethanolamine disalt of DSB. A clear solution resulted with no precipitation. The solution was dried slowly using a TurboVap workstation at 25° C. and 5 psi nitrogen shear followed by drying in a vacuum oven at 35° C.

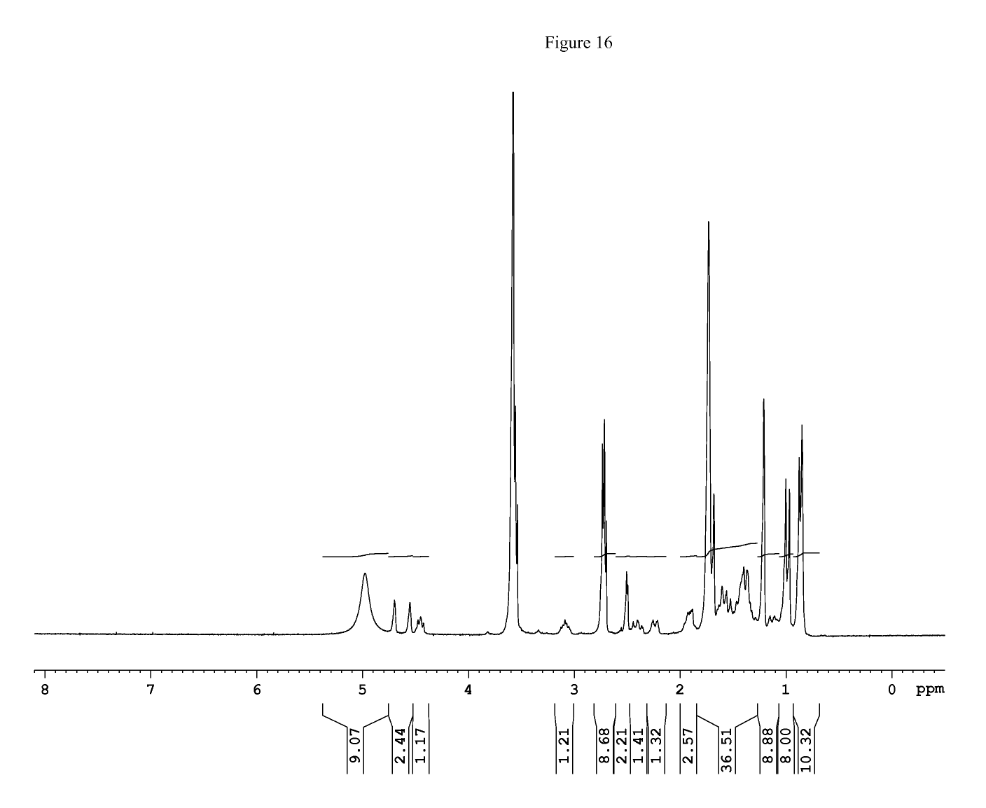

As shown in FIG. 16, ¹H FT-NMR performed in accordance with Method A confirmed that the stoichiometric ratio of the DSB free acid to diethanolamine was 1:2. Chemical shift values for the distinguishing peaks appear about 4.97, 4.74, 4.55, 4.43, 3.59, 3.11, 2.76, 2.50, 2.25, 2.21, 1.93, 1.90, 1.72, 1.68, 1.22, 1.00, 0.97, 0.88, and 0.85 angstroms⁻¹.

The solubility profile of DSB-2 diethanolamine Form I-o, performed in accordance with Method B, is shown in Table 9.

TABLE 9
Solubility Profile of DSB-2 diethanolamine Form I-o Compared
to Free Acid

| | Diethanolamine | |
|---|---|---|
| | Disalt | Free |
| | (μg/mL) | Acid |
| Solvent | Form IO | (μg/mL) |
| Methanol | >40 | 7.5 |
| Ethanol | 10 | 15 |
| 2,2,2- | >40 | 2.5 |
| Trifluoroethanol | | |
| 1-Propanol | 4 | 6.0 |
| 2-Propanol | 4 | 6.0 |
| Water | 4 | <0.5 |

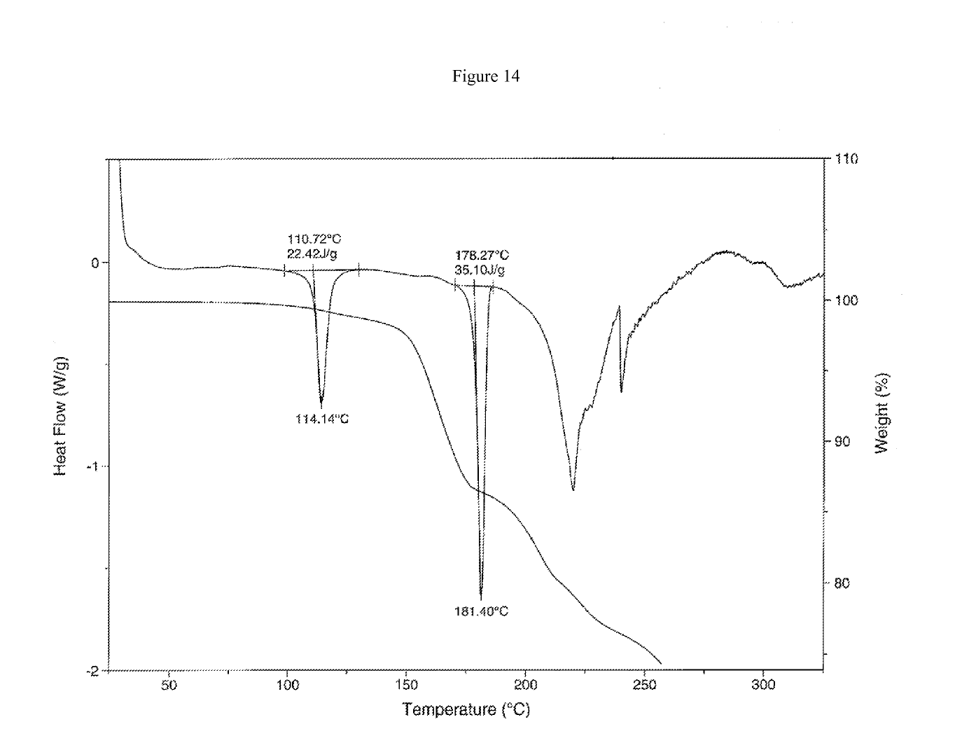

DSC, performed in accordance with Method C and as depicted in FIG. 14, exhibited three endotherms; the first endotherm occurring at about 110° C. did not reveal any changes that corresponded to hot stage observations or thermogravimetric analysis; the second endotherm occurring at about 178° C. corresponds to the partial melt observed with hot stage microscopy; the third endotherm occurring at about 210° C. was attributed to the melting of the remainder of the sample followed immediately by decomposition. TGA, performed in accordance with Method D and as depicted in FIG. 14, demonstrated a mass loss of about 0.4 wt % at about 105° C.

Hot stage microscopy according to Method E indicated that a small fraction of the sample melted at about 176° C. and the remainder of the sample melted over a range of about 214° C. to about 218° C.

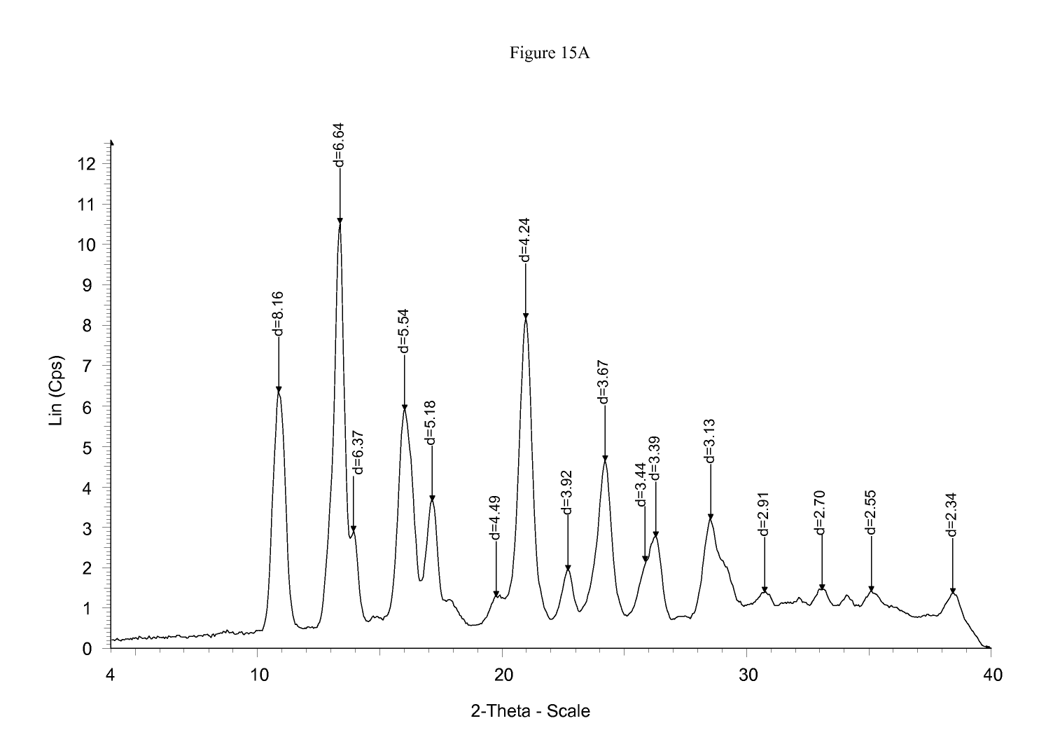

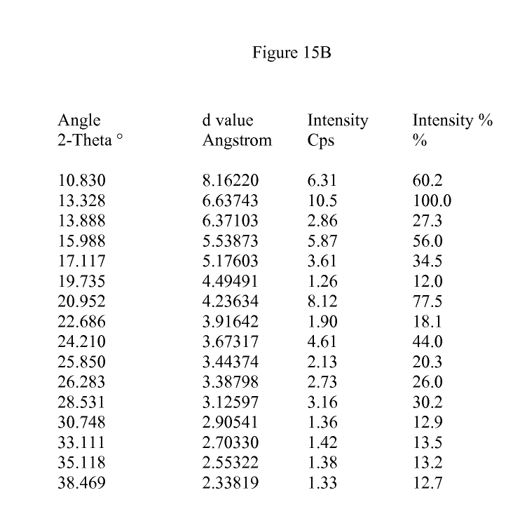

As shown in the PXRD of FIG. 15, performed in accordance with Method G, the DSB-2 diethanolamine Form I-o was a crystalline compound.

In some embodiments, DSB-2 diethanolamine Form I-o is characterized by the X-ray powder diffraction pattern of FIG. 15.

In some embodiments, DSB-2 diethanolamine Form I-o is characterized by an X-ray powder diffraction pattern exhibiting at least one diffraction peak corresponding to d-spacings of about 8.16, 6.64, 6.37, 5.54, 5.18, 4.49, 4.24, 3.91, 3.67, 3.44, 3.39, 3.13, 2.90, 2.70, 2.55, or 2.34 angstroms⁻¹.

In one embodiment the DSB-2 diethanolamine is DSB-2 diethanolamine Form II-o. DSB-2 diethanolamine Form II-o was prepared by recrystallizing DSB-2 diethanolamine Form I-o from 2,2,2-trifluoroethanol, and, optionally, filtering off the remaining liquid to isolate DSB-2 diethanolamine Form II-o.

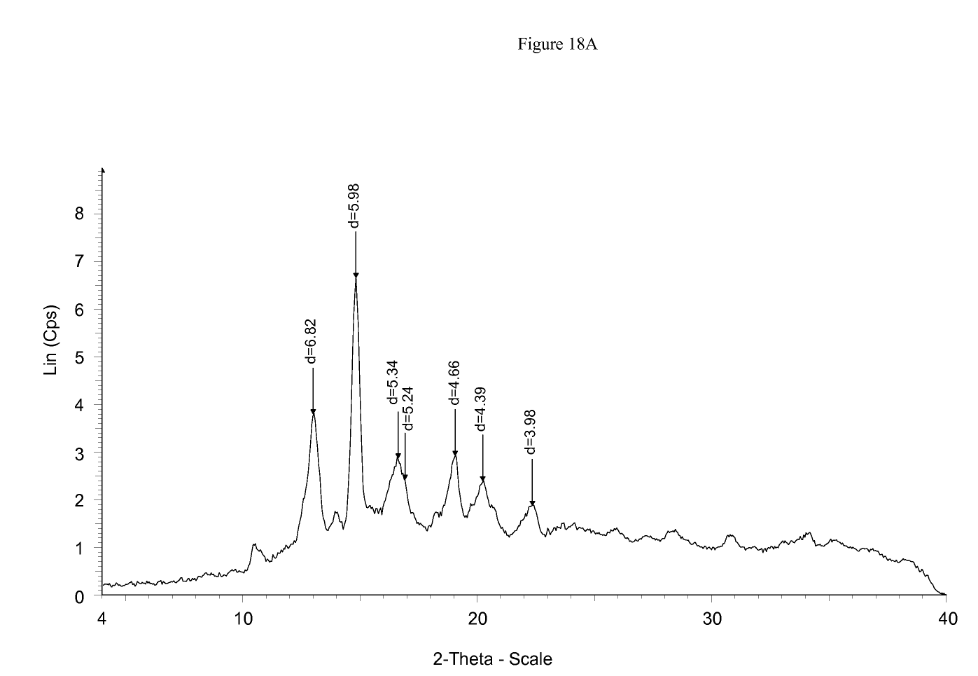

As shown in PXRD of FIG. 18, performed in accordance with Method G, DSB-2 diethanolamine Form II-o is a crystalline compound.

In some embodiments, DSB-2 diethanolamine Form II-o is characterized by the X-ray powder diffraction pattern of FIG. 18.

In some embodiments, DSB-2 diethanolamine Form II-o is characterized by an X-ray powder diffraction pattern exhibiting at least one diffraction peak corresponding to d-spacings of about 8.30, 6.71, 6.45, 5.56, 5.21, 4.27, 3.93, 3.69, 3.41, 3.14, 2.71, or 2.35 angstroms⁻¹. As shown in Tables 10 and 11, in vivo pharmacokinetic studies performed in accordance with Method H demonstrate that the diethanolamine disalt of DSB exhibited surprisingly improved bioavailability and solubility with respect to the free acid of DSB. DSB-2 diethanolamine surprisingly exhibited a 71.3% improvement in bioavailability with respect to the NMG disalt of DSB. Additionally, DSB-2 diethanolamine surprisingly exhibited an 8 fold increase in solubility with respect to the DSB free acid.

TABLE 10
Comparative Mean Plasma Concentrations (ng/mL) of
DSB-2 NMG & DSB-2 Diethanolamine Following Administration
by Oral Gavage to Group 5 Rats

| | Time (h) | | | | | | |
|---|---|---|---|---|---|---|---|
| | 0.083 | 0.25 | 0.5 | 1 | 2 | 4 | 8 |
| Plasma | 491.0 | 3706.7 | 3246.7 | 3236.7 | 1973.7 | 1662.3 | 635.7 |
| Concen- | | | | | | | |
| tration | | | | | | | |
| DSB-2 | | | | | | | |
| NMG | | | | | | | |
| Plasma | 880.7 | 5190.0 | 5743.3 | 5873.3 | 4730.0 | 1830.0 | 1149.3 |
| Concen- | | | | | | | |
| tration | | | | | | | |
| DSB-2 | | | | | | | |
| Di- | | | | | | | |
| ethanol- | | | | | | | |
| amine | | | | | | | |

TABLE 11
Comparative Mean Pharmacokinetic Parameters of DSB-2 NMG
& DSB-2 Diethanolamine

| | AUC_{24 h} | | | % |
|---|---|---|---|---|
| Group-Route | (h * ng/mL) | Cₘₐₓ (ng/mL) | Tₘₐₓ (h) | Bioavailability |
| DSB-2 NMG | 6890.6 | 13785.9 | 0.0 | 100.0 |
| Intravenous | | | | |
| DSB-2 NMG | 18041.1 | 4480.0 | 1.6 | 26.2 |
| Oral Gavage | | | | |
| DSB-2 2 | 30958.7 | 6720.0 | 1.2 | 44.9 |
| Diethanolamine | | | | |
| Oral Gavage | | | | |

Following Administration by Oral Gavage to Group 5 Rats

In some embodiments of the present invention, the DSB-2 diethanolamine drug substance consists of substantially phase pure DSB-2 diethanolamine Form I-o.

In some embodiments of the present invention, the DSB-2 diethanolamine drug substance comprises at least about 90% DSB-2 diethanolamine Form I-o relative to all other solid state forms of DSB-2 diethanolamine present in the DSB-2 diethanolamine drug substance.

In some embodiments of the present invention, the DSB-2 diethanolamine drug substance comprises at least about 75% DSB-2 diethanolamine Form I-o relative to all other solid state forms of DSB-2 diethanolamine present in the DSB-2 diethanolamine drug substance.

In some embodiments of the present invention, the DSB-2 diethanolamine drug substance comprises at least a detectable amount of DSB-2 diethanolamine Form I-o relative to all other solid state forms of DSB-2 diethanolamine present in the DSB-2 diethanolamine drug substance.

In some embodiments of the present invention, the DSB-2 diethanolamine drug substance consists of substantially phase pure DSB-2 diethanolamine Form II-o.

In some embodiments of the present invention, the DSB-2 diethanolamine drug substance comprises at least about 90% DSB-2 diethanolamine Form II-o relative to all other solid state forms of DSB-2 diethanolamine present in the DSB-2 diethanolamine drug substance.

In some embodiments of the present invention, the DSB-2 diethanolamine drug substance comprises at least about 75% DSB-2 diethanolamine Form II-o relative to all other solid state forms of DSB-2 diethanolamine present in the DSB-2 diethanolamine drug substance.

In some embodiments of the present invention, the DSB-2 diethanolamine drug substance comprises at least a detectable amount of DSB-2 diethanolamine Form II-o relative to all other solid state forms of DSB-2 diethanolamine present in the DSB-2 diethanolamine drug substance.

One embodiment of the present invention comprises a pharmaceutical composition comprising a diethanolamine salt of DSB, such as the di-diethanolamine salt of DSB, and a pharmaceutically acceptable excipient.

One embodiment of the present invention comprises a method of preparing a diethanolamine salt of DSB. In one embodiment of the invention, the method of preparing the salt comprises mixing diethanolamine and DSB in an aqueous solution to provide DSB-2 diethanolamine Form I-o.

One embodiment of the present invention comprises a method of using a pharmaceutical composition that comprises a diethanolamine salt of DSB, such as DSB-2 diethanolamine Form I-o or DSB-2 diethanolamine Form II-o, for treating, in a human subject, a retroviral infection, such as HIV.

### Example 5

### Preparation and Characterization of DSB Diethylamine Disalt

One embodiment of the present invention comprises a diethylamine salt of DSB. In one embodiment the diethylamine salt of DSB is the bi-diethylamine salt of DSB (“DSB-2 diethylamine”). DSB-2 diethylamine has about two diethylamine molecules per DSB molecule; has a molecular formula of about C₃₆H₅₆O₆.[C₄H₁₁N]₂, a molecular weight of about 731.1064 and has the following structural formula:

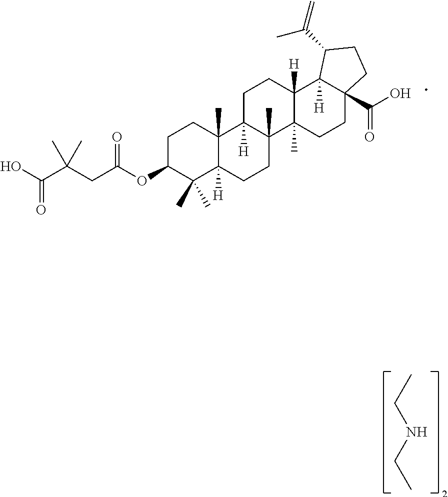

In one embodiment the DSB-2 diethylamine is DSB-2 diethylamine Form I-y. DSB-2 diethylamine Form I-y was prepared by dissolving DSB free acid in ethanol. The diethylamine was dissolved in water. Two equivalents of the diethylamine solution were mixed with one equivalent of the DSB solution to form DSB-2 diethylamine Form I-y. A clear solution resulted with no precipitation. The solution was dried slowly using a TurboVap workstation at 25° C. and 5 psi nitrogen shear followed by drying in a vacuum oven at 35° C. The solids were homogenized and characterized for crystallinity by PXRD according to Method G, thermal properties by DSC and TGA according to Methods C and D respectively. Recrystallization of DSB-2 diethylamine Form I-y in ethanol, 2,2,2-trifluoroethanol, 1-propanol, 2-propanol, water, or acetone provided DSB-2 diethylamine Form I-y.

As shown in FIG. 21, ¹H FT-NMR performed in accordance with Method A confirmed that the stoichiometric ratio of the DSB free acid to diethylamine was 1:2. Chemical shift values for the distinguishing peaks appear about 5.77, 4.76, 4.58, 4.49, 4.47, 4.46, 4.42, 3.60, 3.10, 2.63, 2.61, 2.60, 2.59, 2.51, 2.43, 2.41, 2.38, 2.27, 2.22, 1.94, 1.90, 1.75, 1.71, 1.60, 1.57, 1.54, 1.40, 1.36, 1.20, 1.07, 1.05, 1.03, 0.99, 0.97, 0.83, and 0.84 ppm.

The solubility profile of the DSB-2 diethylamine Form I-y, performed in accordance with Method B, is shown in Table 12.

TABLE 12
Solubility Profile of DSB-2 diethylamine Form I-y Compared to
Free Acid

| | Diethylamine | |
|---|---|---|
| | Disalt | Free |
| | Form I | Acid |
| Solvent | (μg/mL) | (μg/mL) |
| Methanol | >40 | 7.5 |
| Ethanol | 10 | 15 |
| 2,2,2- | >40 | 2.5 |
| Trifluoroethanol | | |
| 1-Propanol | 10 | 6.0 |
| 2-Propanol | 4 | 6.0 |
| Water | <0.5 | <0.5 |

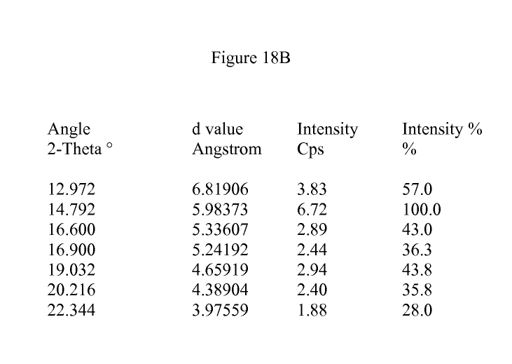

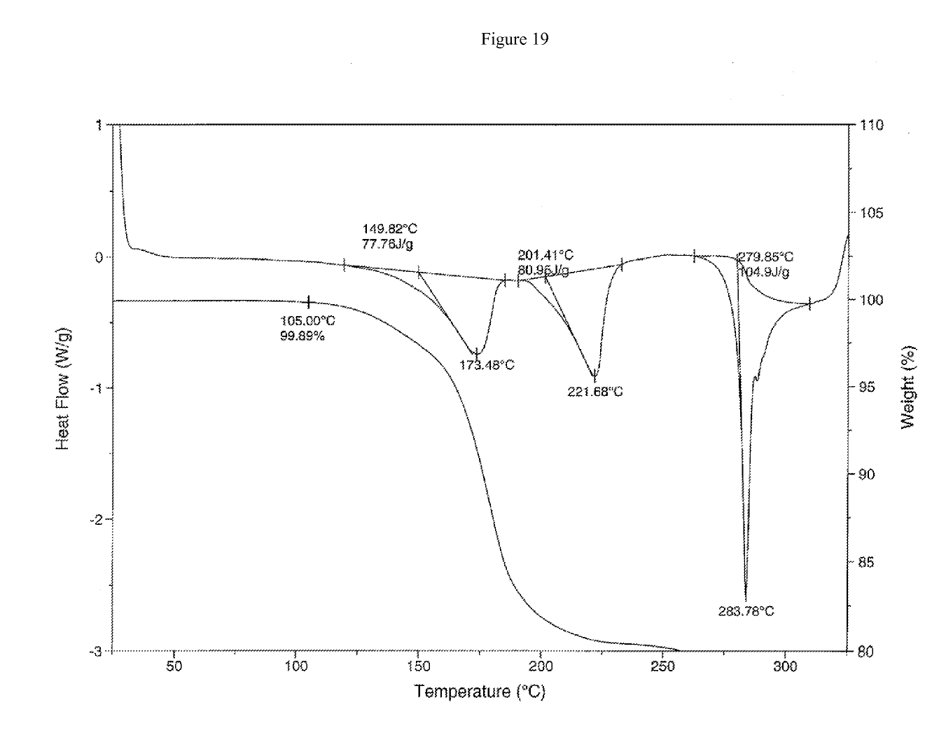

DSC performed in accordance with Method C exhibited three endotherms; the first endotherm occurring at about 150° C. corresponds with about a 20% weight loss as indicated by TGA; the second endotherm occurring at about 220° C. corresponds with the sample melting; the third endotherm corresponds with the decomposition of the free acid. The sample lost about 4 wt % at about 105° C. The DSC/TGA overlay thermogram is shown in FIG. 19.

Hot stage microscopy according to Method E indicated that the sample melted over a range of about 227° C. to about 229° C.

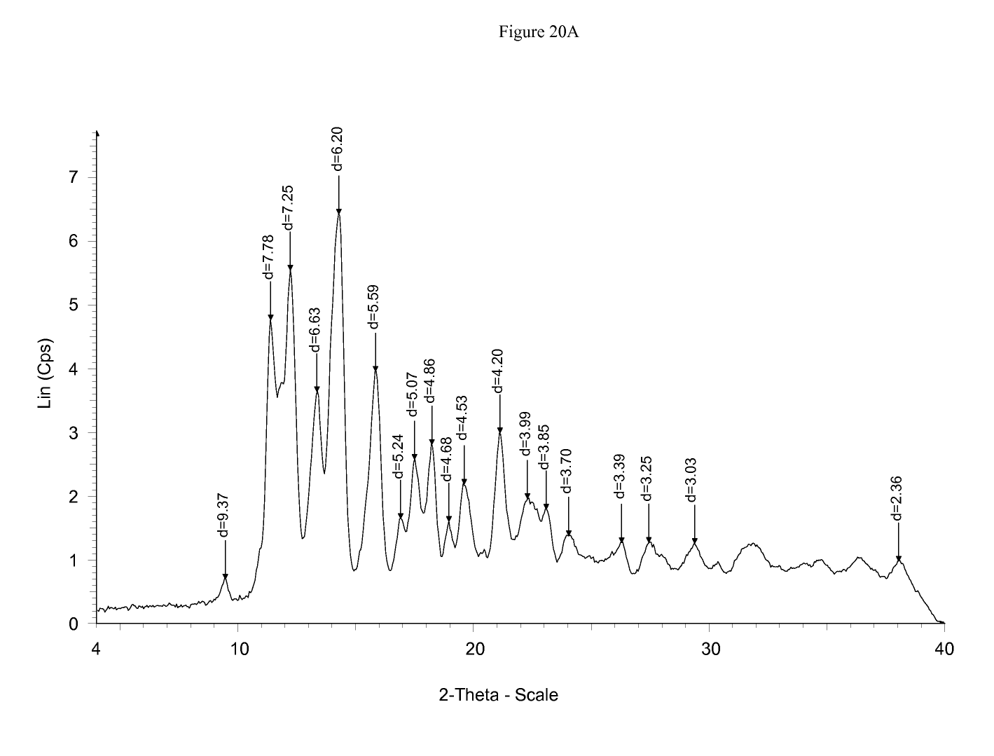

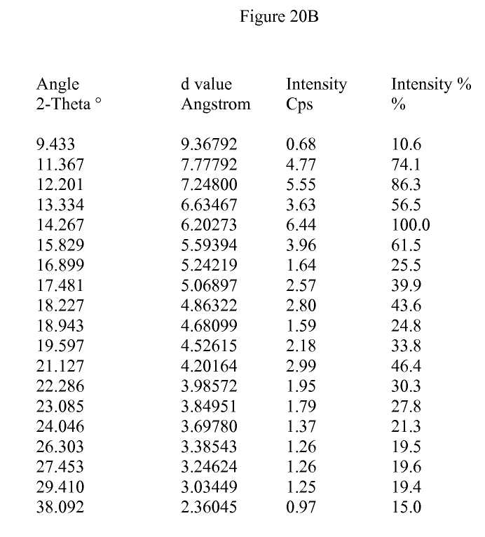

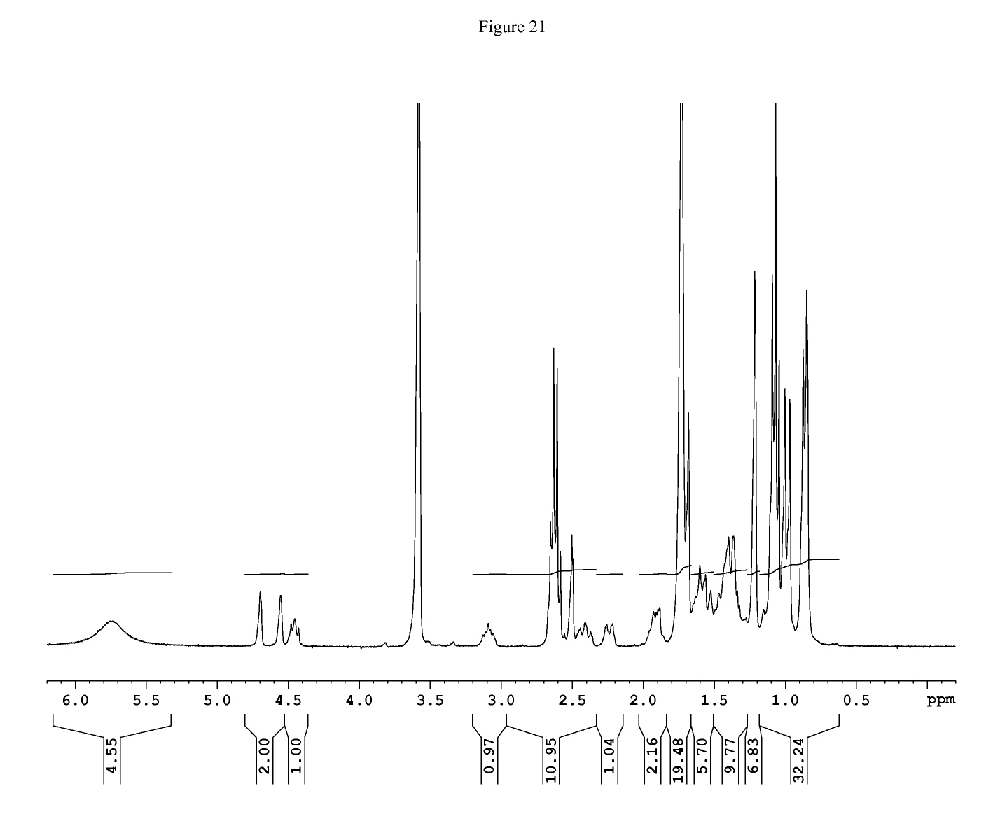

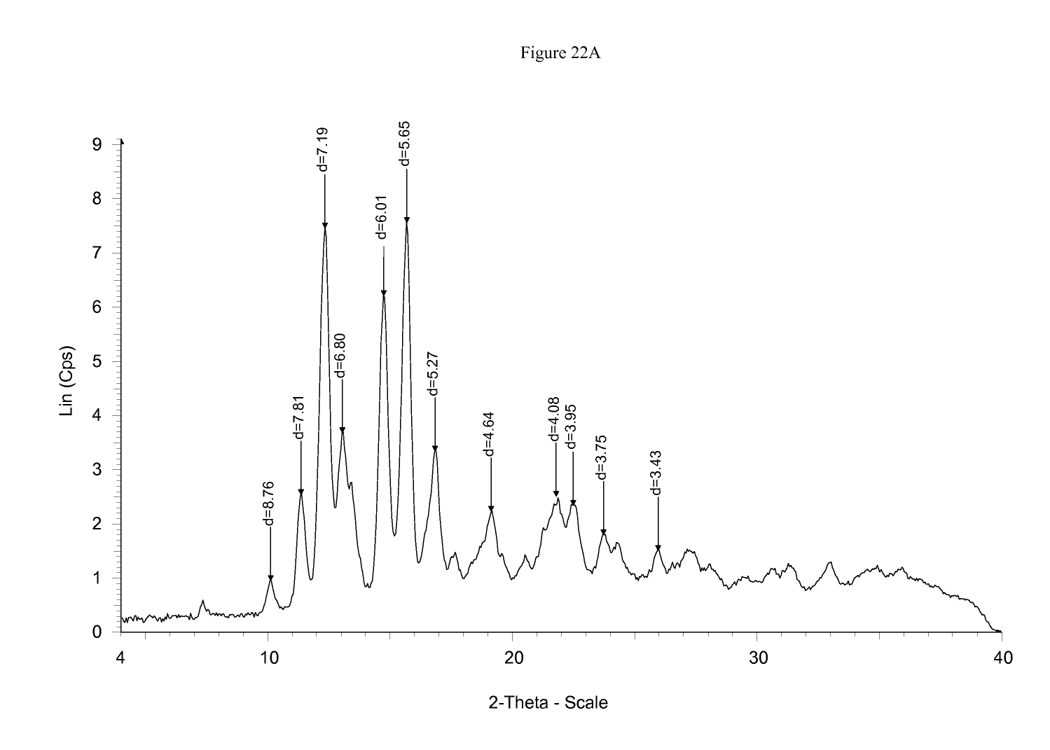

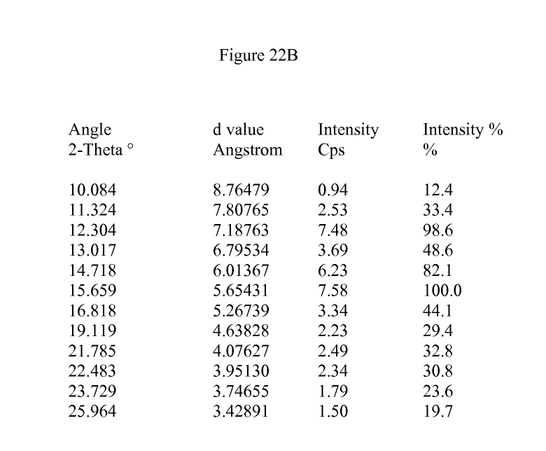

As shown in the PXRD of FIG. 20, performed in accordance with Method G, the DSB-2 diethylamine Form I-y was a crystalline compound.

In some embodiments, DSB-2 diethylamine Form I-y is characterized by the X-ray powder diffraction pattern of FIG. 20.

In some embodiments, DSB-2 diethylamine Form I-y is characterized by an X-ray powder diffraction pattern exhibiting at least one diffraction peak corresponding to d-spacings of about 9.37, 7.78, 7.25, 6.63, 6.20, 5.59, 5.24, 5.07, 4.86, 4.68, 4.53, 4.20, 3.99, 3.85, 3.70, 3.39, 3.25, 3.03, or 2.36 angstroms⁻¹.

As shown in Tables 13 and 14, in vivo pharmacokinetic studies performed in accordance with Method H demonstrate a surprisingly high Cₘₐₓ and a surprisingly small Tₘₐₓ value, suggesting that this compound could be useful in situations where it is desirable to reach a high plasma concentration in a short amount of time.

TABLE 13
Comparative Mean Plasma Concentrations (ng/mL) of DSB-2 NMG &
DSB-2 Diethylamine Following Administration to Group 6 Rats

| | Time (h) | | | | | | |
|---|---|---|---|---|---|---|---|
| | 0.083 | 0.25 | 0.5 | 1 | 2 | 4 | 8 |
| Plasma | 491.0 | 3706.7 | 3246.7 | 3236.7 | 1973.7 | 1662.3 | 635.7 |
| Concen- | | | | | | | |
| tration | | | | | | | |
| DSB-2 | | | | | | | |
| NMG | | | | | | | |
| Plasma | 352.7 | 4576.7 | 7420.0 | 6873.3 | 3750.0 | 1886.7 | 233.7 |
| Concen- | | | | | | | |
| tration | | | | | | | |
| DSB-2 | | | | | | | |
| Diethyl- | | | | | | | |
| amine | | | | | | | |

TABLE 14
Comparative Mean Pharmacokinetic Parameters of DSB-2 NMG
& DSB-2 Diethylamine Following Administration to Group 6 Rats

| | AUC_{24 h} | | | % |
|---|---|---|---|---|
| Group-Route | (h * ng/mL) | Cₘₐₓ (ng/mL) | Tₘₐₓ (h) | Bioavailability |
| DSB-2 NMG | 6890.6 | 13785.9 | 0.0 | 100.0 |
| Intravenous | | | | |
| DSB-2 NMG | 18041.1 | 4480.0 | 1.6 | 26.2 |
| Oral Gavage | | | | |
| DSB-2 2 | 21172.3 | 7453.3 | 0.67 | 30.7 |
| Diethylamine | | | | |
| Oral Gavage | | | | |

In one embodiment the DSB-2 diethylamine is DSB-2 diethylamine Form II-y. Recrystallization is achieved by dissolving the DSB-2 diethylamine Form I-y in a minimum volume of methylene chloride to create a mixture, warming the mixture to create a warmed solution, removing any insoluble impurities by filtration, slowly cooling the warmed solution to crystallize DSB-2 diethylamine Form II-y, and, optionally, filtering off the remaining liquid to isolate the recrystallized DSB-2 diethylamine Form II-y.

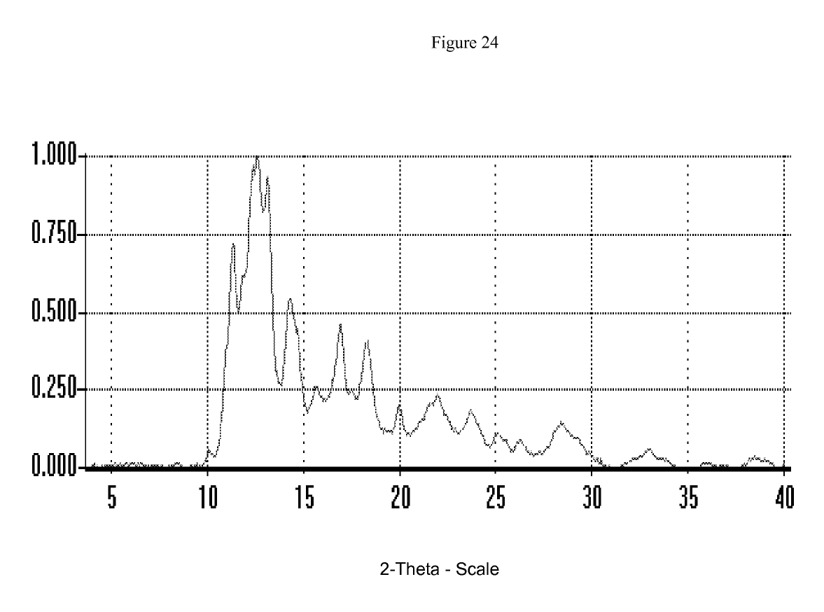

In some embodiments, DSB-2 diethylamine Form II-y is characterized by the X-ray powder diffraction pattern of FIG. 24.

In some embodiments, DSB-2 diethylamine Form II-y is characterized by an X-ray powder diffraction pattern exhibiting at least one diffraction peak corresponding to d-spacings of about 8.76, 7.81, 7.19, 6.79, 6.01, 5.65, 5.27, 4.63, 4.08, 3.95, 3.75, or 3.43 angstroms⁻¹.

In one embodiment the DSB-2 diethylamine is DSB-2 diethylamine Form III-y. DSB-2 diethylamine Form III-y was prepared by dissolving DSB-2 diethylamine Form I-y in a minimum volume of methanol to create a mixture, warming the mixture to create a warmed solution, removing any insoluble impurities by filtration, slowly cooling the warmed solution to crystallize DSB-2 diethylamine Form III-y, and, optionally, filtering off the remaining liquid to isolate the DSB-2 diethylamine Form III-y.

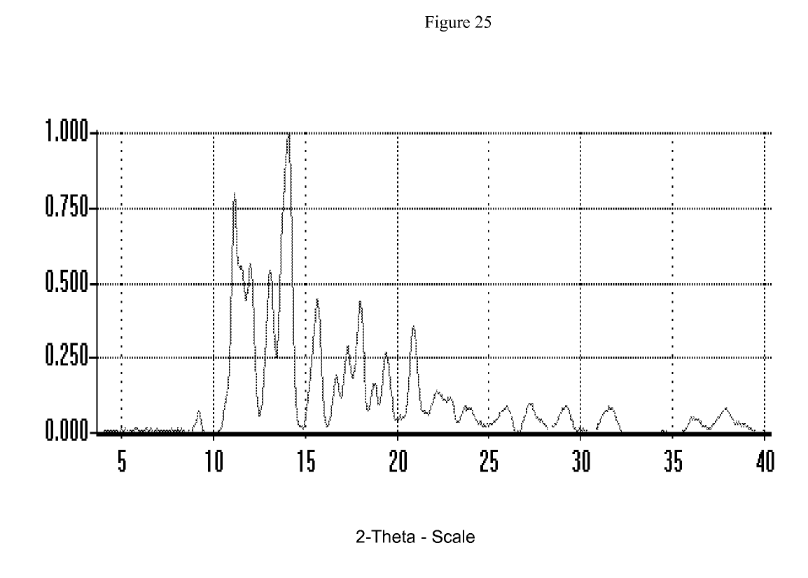

In some embodiments, DSB-2 diethylamine Form III-y is characterized by the X-ray powder diffraction pattern of FIG. 25.

In some embodiments, DSB-2 diethylamine Form III-y is characterized by an X-ray powder diffraction pattern exhibiting at least one diffraction peak corresponding to d-spacings of about 8.52, 7.81, 7.14, 6.81, 5.98, 5.67, 5.32, 4.88, 4.66, 4.39, 3.98, 3.44, 3.26, 2.91, or 2.31 angstroms⁻¹.

In one embodiment the DSB-2 diethylamine is DSB-2 diethylamine Form IV-y. DSB-2 diethylamine Form IV-y was prepared by dissolving DSB-2 diethylamine Form I-y in a minimum volume of N,N-dimethylformamide or ethyl acetate to create a mixture, warming the mixture to create a warmed solution, removing any insoluble impurities by filtration, slowly cooling the warmed solution to crystallize DSB-2 diethylamine Form IV-y, and, optionally, filtering off the remaining liquid to isolate the recrystallized DSB-2 diethylamine Form IV-y.

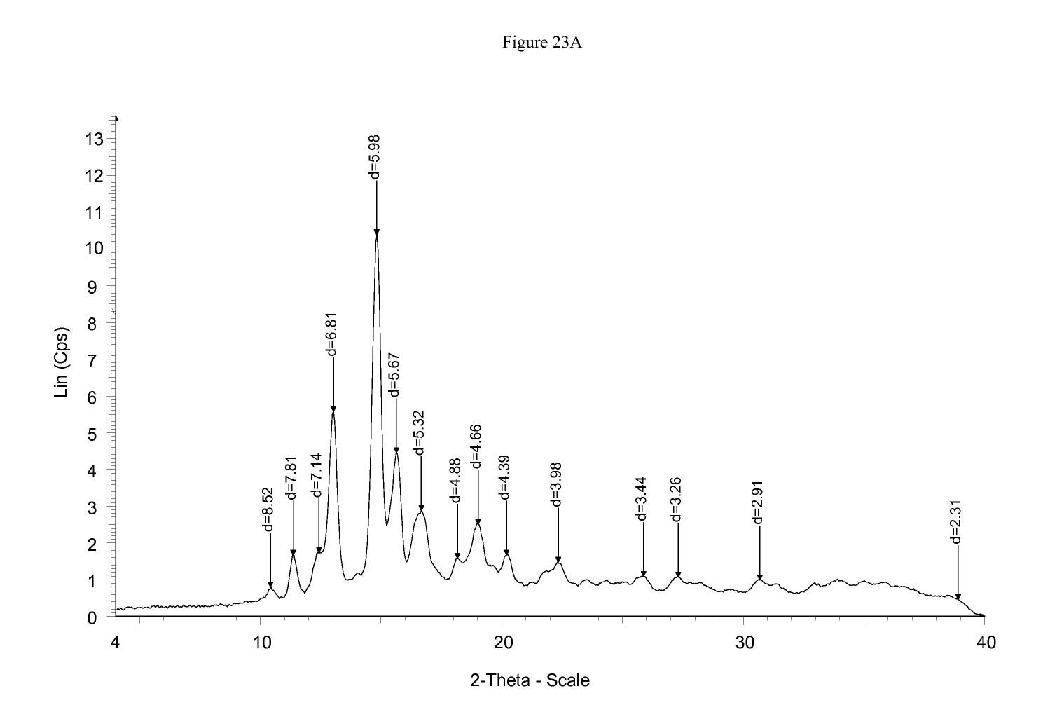

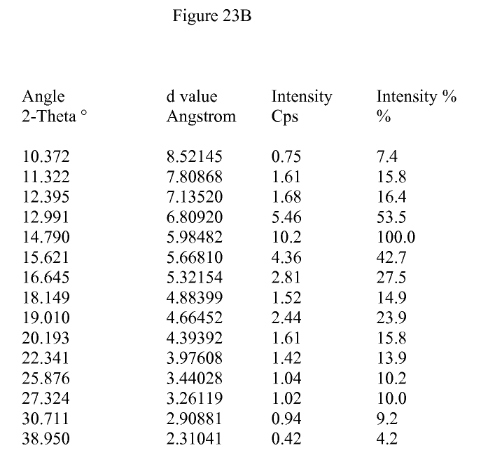

As shown in PXRD of FIGS. 23, 24 and 52, performed in accordance with Method G, DSB-2 diethylamine Form II-y, DSB-2 diethylamine Form III-y, and DSB-2 diethylamine Form IV-y are crystalline compounds.

In some embodiments of the present invention, the DSB-2 diethylamine drug substance consists of substantially phase pure DSB-2 diethylamine Form I-y.

In some embodiments of the present invention, the DSB-2 diethylamine drug substance comprises at least about 90% DSB-2 diethylamine Form I-y relative to all other solid state forms of DSB-2 diethylamine present in the DSB-2 diethylamine drug substance.

In some embodiments of the present invention, the DSB-2 diethylamine drug substance comprises at least about 75% DSB-2 diethylamine Form I-y relative to all other solid state forms of DSB-2 diethylamine present in the DSB-2 diethylamine drug substance.

In some embodiments of the present invention, the DSB-2 diethylamine drug substance comprises at least a detectable amount of DSB-2 diethylamine Form I-y relative to all other solid state forms of DSB-2 diethylamine present in the DSB-2 diethylamine drug substance.

In some embodiments of the present invention, the DSB-2 diethylamine drug substance consists of substantially phase pure DSB-2 diethylamine Form II-y.

In some embodiments of the present invention, the DSB-2 diethylamine drug substance comprises at least about 90% DSB-2 diethylamine Form II-y relative to all other solid state forms of DSB-2 diethylamine present in the DSB-2 diethylamine drug substance.

In some embodiments of the present invention, the DSB-2 diethylamine drug substance comprises at least about 75% DSB-2 diethylamine Form II-y relative to all other solid state forms of DSB-2 diethylamine present in the DSB-2 diethylamine drug substance.

In some embodiments of the present invention, the DSB-2 diethylamine drug substance comprises at least a detectable amount of DSB-2 diethylamine Form II-y relative to all other solid state forms of DSB-2 diethylamine present in the DSB-2 diethylamine drug substance.

In some embodiments of the present invention, the DSB-2 diethylamine drug substance consists of substantially phase pure DSB-2 diethylamine Form III-y.

In some embodiments of the present invention, the DSB-2 diethylamine drug substance comprises at least about 90% DSB-2 diethylamine Form III-y relative to all other solid state forms of DSB-2 diethylamine present in the DSB-2 diethylamine drug substance.

In some embodiments of the present invention, the DSB-2 diethylamine drug substance comprises at least about 75% DSB-2 diethylamine Form III-y relative to all other solid state forms of DSB-2 diethylamine present in the DSB-2 diethylamine drug substance.

In some embodiments of the present invention, the DSB-2 diethylamine drug substance comprises at least a detectable amount of DSB-2 diethylamine Form III-y relative to all other solid state forms of DSB-2 diethylamine present in the DSB-2 diethylamine drug substance.

In some embodiments of the present invention, the DSB-2 diethylamine drug substance consists of substantially phase pure DSB-2 diethylamine Form IV-y.

In some embodiments of the present invention, the DSB-2 diethylamine drug substance comprises at least about 90% DSB-2 diethylamine Form IV-y relative to all other solid state forms of DSB-2 diethylamine present in the DSB-2 diethylamine drug substance.

In some embodiments of the present invention, the DSB-2 diethylamine drug substance comprises at least about 75% DSB-2 diethylamine Form IV-y relative to all other solid state forms of DSB-2 diethylamine present in the DSB-2 diethylamine drug substance.

In some embodiments of the present invention, the DSB-2 diethylamine drug substance comprises at least a detectable amount of DSB-2 diethylamine Form IV-y relative to all other solid state forms of DSB-2 diethylamine present in the DSB-2 diethylamine drug substance.

One embodiment of the present invention comprises a pharmaceutical composition comprising a diethylamine salt of DSB, such as a bis-diethylamine salt of DSB, and a pharmaceutically acceptable excipient.

One embodiment of the present invention comprises a method of preparing a DSB-2 diethylamine Form I-y. In one embodiment of the invention, the method of preparing the salt comprises mixing diethylamine and DSB in an aqueous solution to provide 3-O-(3′,3′-dimethylsuccinyl)betulinic acid, bis-diethylamine salt.

One embodiment of the present invention comprises a method of using a pharmaceutical composition that comprises a diethylamine salt of DSB, such as the bis-diethylamine salt of DSB for treating, in a human subject, a retroviral infection, such as HIV.

### Pharmaceutical Compositions

The present invention further provides a pharmaceutical composition comprising a therapeutically effective amount of DSB salt of the present invention and a pharmaceutically acceptable carrier.

The present invention yet further provides a pharmaceutical composition comprising a therapeutically effective amount of a DSB salt of the present invention and one, two, three, four, five or six agents selected from the group consisting of a HIV protease inhibitor, a HIV reverse transcriptase inhibitor, an HIV entry or fusion inhibitor, an HIV integrase inhibitor and an HIV maturation inhibitor, and a pharmaceutically acceptable carrier.

### Unit Dosages

Mass references described in this application refer to mass of the free acid equivalent unless otherwise.

Illustrative dosage unit forms of the pharmaceutical compositions can typically contain about, 100, 200, 250, 300, 350, 400, 450, or 500 mg of a DSB salt of the present invention. Preferred dosage unit forms contain about 200, 300, 400, or 500 mg of a DSB salt of the present invention. The dosage unit form can be selected to accommodate the desired frequency of administration used to achieve the specified daily dosage. The amount of the unit dosage form of the pharmaceutical composition that is administered and the dosage regimen for treating the condition or disorder depends on a variety of factors, including the age, weight, sex and medical condition of the subject, the severity of the condition or disorder, the route and frequency of administration, and thus can vary widely, as is well known.

Where it is desired to formulate dosage units consisting of less than the therapeutically effective amount, multiple dosage units, each containing smaller amounts of the DSB salt, can be administered to constitute the daily dose.

The amount of active ingredient that may be combined with the carrier materials to produce a single dosage form will vary depending upon the host treated and the particular mode of administration.

It will be understood, however, that the specific dose level for any particular patient will depend upon a variety of factors including the activity of the specific compound employed, the age, body weight, general health, sex, diet, time of administration, route of administration, rate of excretion, drug combination, and the severity of the particular disease undergoing therapy.

The compounds of the present invention may be administered orally, parenterally, sublingually, rectovaginally, topically, transmucosally, transdermally, [or through lisosomes] in dosage unit formulations optionally comprising conventional nontoxic pharmaceutically acceptable carriers, adjuvants, or vehicles as desired.

“Formulations suitable for systemic administration” means formulations which are in a form suitable to be administered systemically to a patient. Systemic administration can be achieved by oral delivery, parenteral delivery, transmucosal delivery, transdermal delivery, rectovaginal delivery or liposomal delivery.

“Formulations suitable for oral administration” means formulations which are in a form suitable to be administered orally to a patient. In some embodiments, the oral formulation is intended to be absorbed in the gastric or intestinal cavities. The formulations may be presented as discrete units such as capsules, cachets or tablets each containing a predetermined amount of the active ingredient; as a powder or granules; as solution or a suspension in an aqueous liquid or a non-aqueous liquid; or as an oil-in-water liquid emulsion or a water-in-oil liquid emulsion. The active ingredient may also be presented as a bolus, electuary or paste. Solid dosage forms for oral administration may include capsules, tablets, pills, powders, and granules. In such solid dosage forms, the active compound may be admixed with at least one inert diluent such as sucrose, lactose or starch. Such dosage forms may also comprise, as is normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coating. Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, and sweetening, flavoring, and perfuming agents. In some embodiments, the oral formulation is intended to be absorbed at least in part in the oral cavity including the lips, the inside lining of the lips and cheeks (buccal mucosa), the teeth, the gums (gingivae), the tongue, the floor of the mouth below the tongue, the bony roof of the mouth (hard palate), the area behind the wisdom teeth (retromolar trigone), and the salivary glands. Formulations suitable for topical administration in the mouth include lozenges comprising the active ingredient in a flavored basis, for example sucrose and acacia or tragacanth; pastilles comprising the active ingredient in an inert basis such as gelatin and glycerin, or sucrose and acacia; and mouthwashes comprising the active ingredient in a suitable liquid carrier.

“Formulations suitable for parenteral administration” means formulations which are in a form suitable to be administered parenterally to a patient. The term “parenteral” as used herein includes subcutaneous delivery, intravenous delivery, and intramuscular delivery. In some embodiments of the present invention, the formulations comprise emulsions, suspensions, aqueous or non-aqueous injection solutions. Injectable formulations, for example sterile injectable aqueous or oleagenous suspensions, may be formulated according to the known art using suitable dispersing or wetting agents and suspending agents, thickening agents, anti-oxidants, buffers, bacteriostats, and solutes which render the formulation isotonic. In preferred embodiments formulations suitable for parenteral administration have a pH adjusted to be compatible with the blood of the intended recipient. The sterile injectable formulation may also be a sterile injectable solution or suspension in a nontoxic parenterally acceptable diluent or solvent, for example, as a solution in 1,3-propanediol. Among the acceptable vehicles and solvents that may be employed are physiologically compatible buffers such as water, Hank's solution, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables. Some embodiments of the present invention comprise lyophilized formulations. In some embodiments of the present invention, the compounds are formulated in solid form and redissolved or suspended immediately prior to use.

“Formulations suitable for topical administration” means formulations which are in a form suitable to be administered topically to a patient. The formulation may be presented as a topical ointment, salves, powders, alcohol based gels, water based gels, creams, as is generally known in the art, or incorporated into a matrix base for application in a patch, which would allow a controlled release of compound through the transdermal barrier. When formulated in an ointment, the active ingredients may be employed with either a paraffinic or a water-miscible ointment base. Alternatively, the active ingredients may be formulated in a cream with an oil-in-water cream base. In some embodiments, the transmucosal or transdermal formulation comprises a penetrant appropriate to the barrier to be permeated by at least one active ingredient of the formulation. Such penetrants are generally known in the art, and include, for example, bile salts and fusidic acid derivatives for transmucosal administration. In addition, detergents may be used to facilitate permeation.

“Formulations suitable for rectovaginal administration” means formulations which are in a form suitable to be administered to the rectum or vagina of a patient.

“Formulations suitable for rectal administration” means formulations which are in a form suitable to be administered rectally to a patient. The rectal formulation is preferably administered in the form of suppositories which can be prepared by mixing the compounds useful according to this invention with suitable non-irritating excipients or carriers such as cocoa butter, a poly(ethylene glycol) or a suppository wax, which are solid at ordinary temperatures but liquid at body temperature and therefore, melt in the rectum or vaginal cavity and release the active component.

“Formulations suitable for vaginal administration” means formulations which are in a form suitable to be administered vaginally to a patient. The formulation may be presented as pessaries, tampons, creams, gels, pastes, foams or spray formulations containing in addition to the active ingredient such carriers as are known in the art to be appropriate.

The compounds of the present invention can also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any nontoxic, physiologically acceptable and metabolizable lipid capable of forming liposomes can be used. The present compositions in liposome form can contain, in addition to the compound of the present invention, stabilizers, preservatives, excipients, and the like. The preferred lipids are the phospholipids and phosphatidyl cholines (lecithins), both natural and synthetic.

### Form of Pharmaceutical Compositions

The pharmaceutical compositions of the present invention comprise a DSB salt of the present invention in association with one or more non-toxic, pharmaceutically acceptable carriers, excipients or adjuvants (collectively referred to herein as “carrier materials”). The carrier materials are acceptable in the sense of being compatible with the other ingredients of the composition and are not deleterious to the recipient. The pharmaceutical compositions of the present invention can be adapted for administration by any suitable route by selection of appropriate carrier materials and a dosage of a DSB salt of the present invention effective for the treatment intended. For example, these compositions can be prepared in a form suitable for administration orally, intravascularly, intraperitoneally, subcutaneously, intramuscularly or rectally. Accordingly, the carrier material employed can be a solid or a liquid, or both, and is preferably formulated with the compound as a unit-dose composition, for example, a tablet, which can contain from about 1% to about 95%, preferably about 25% to about 70%, more preferably about 40% are to about 60%, and still more preferably about 20%, by weight of a DSB salt of the present invention. Such pharmaceutical compositions of the invention can be prepared by any of the well known techniques of pharmacy, consisting essentially of admixing the components.

### Oral Administration

For oral administration, the pharmaceutical composition can contain a desired amount of a DSB salt of the present invention and be in the form of, for example, a tablet, a hard or soft capsule, a lozenge, a cachet, a dispensable powder, granules, a suspension, an elixir, a liquid, or any other form reasonably adapted for oral administration. Such a pharmaceutical composition is preferably made in the form of a discrete dosage unit containing a predetermined amount of a DSB salt of the present invention, such as tablets or capsules. Such oral dosage forms can further comprise, for example, buffering agents. Tablets, pills and the like additionally can be prepared with enteric coatings. Unit dosage tablets or capsules are preferred.

Pharmaceutical compositions suitable for buccal (sub-lingual) administration include, for example, lozenges comprising a DSB salt of the present invention in a flavored base, such as sucrose, and acacia or tragacanth, and pastilles comprising a DSB salt of the present invention in an inert base such as gelatin and glycerin or sucrose and acacia.

Liquid dosage forms for oral administration can include pharmaceutically acceptable emulsions, solutions, suspensions, syrups, and elixirs containing inert diluents commonly used in the art, such as water or a cyclodextrin. Such compositions can also comprise, for example, wetting agents, emulsifying and suspending agents, and sweetening, flavoring, and perfuming agents. Examples of suitable liquid dosage forms include, but are not limited, aqueous solutions comprising a DSB salt of the present invention and β-cyclodextrin or a water soluble derivative of β-cyclodextrin such as sulfobutyl ether β-cyclodextrin, heptakis-2,6-di-O-methyl-β-cyclodextrin, hydroxypropyl-β-cyclodextrin, or dimethyl-(3-cyclodextrin.

### Parenteral Administration

The pharmaceutical compositions of the present invention can also be administered parenterally (subcutaneous, intravenous, or intramuscular). Such injectable compositions can employ, for example, saline, dextrose, or water as a suitable carrier material. The pH value of the composition can be adjusted, if necessary, with suitable acid, base, or buffer. Suitable bulking, dispersing, wetting or suspending agents, including mannitol and poly(ethylene glycol)s (such as PEG 400), can also be included in the composition. A suitable parenteral composition can also include a DSB salt of the present invention in injection vials. Aqueous solutions can be added to dissolve the composition prior to injection.

### Rectovaginal Administration

The pharmaceutical compositions can be administered either rectally or vaginally. Illustrative pharmaceutical compositions are administered in the form of a suppository or a pessary. In some embodiments, the rectovaginal formulations comprise a DSB salt of the present invention in a total amount of, for example, 0.075 to 30% w/w, preferably 0.2 to 20% w/w and most preferably 0.4 to 15% w/w. Carrier materials such as cocoa butter, theobroma oil, and other oil and poly(ethylene glycol) suppository bases can be used in such compositions. Other carrier materials such as coatings (for example, hydroxypropylmethylcellulose film coating) and disintegrants (for example, croscarmellose sodium and cross-linked povidone) can also be employed if desired.

As indicated above, these pharmaceutical compositions can be prepared by any suitable method of pharmacy which includes the step of bringing into association a DSB salt of the present invention and at least one carrier material. In general, the compositions are prepared by uniformly and intimately admixing the active compound with a liquid or finely divided solid carrier, or both, and then, optionally coating the admixture, and then, optionally shaping the product. For example, a tablet can be prepared by compressing or molding a powder or granules of the compound, optionally with one or more accessory ingredients. Compressed tablets can be prepared by compressing, in a suitable machine, the compound in a free-flowing form, such as a powder or granules optionally mixed with a binding agent, lubricant, inert diluent or surface active/dispersing agent. Molded tablets can be made by molding, in a suitable machine, the powdered compound moistened with an inert liquid diluent.

### Carrier Materials

As noted above, for therapeutic purposes, the pharmaceutical compositions of the present invention comprise a DSB salt of the present invention in a desired amount in combination with at least one pharmaceutically acceptable carrier material appropriate to the indicated route of administration. It is understood in the art that certain carrier materials may provide a plurality of functions, for example hydroxypropylmethylcellulose may function as both a water retention agent and as an emulsifier; as such the inclusion of any particular excipient as a member of one class is not intended to limit other classes to its exclusion.

Oral dosage forms of the pharmaceutical compositions of the present invention preferably comprise a DSB salt of the present invention in a desired amount admixed with one or more carrier materials selected from the group consisting of diluents, disintegrants, binding agents and adhesives, wetting agents, lubricants, and anti-adherents.

Preferably, oral dosage forms of the present invention are tableted or encapsulated for convenient administration.

Injectable dosage forms preferably are adapted for parenteral injection. Preferably, these dosage forms comprise a DSB salt of the present invention in aqueous or non-aqueous isotonic sterile injection solutions or suspensions, such as a DSB salt of the present invention suspended or dissolved in water, poly(ethylene glycol), propylene glycol, ethanol, corn oil, cottonseed oil, peanut oil, sesame oil, benzyl alcohol, sodium chloride, and/or various buffers. These solutions and suspensions can be prepared from sterile powders or granules having one or more of the carriers or diluents mentioned for use in the formulations for oral administration.

The selection and combination of carrier materials used in the pharmaceutical compositions of the present invention provides compositions exhibiting improved performance with respect to, among other properties, safety, efficacy, dissolution profile, disintegration profile, bioavailability, clearance times, stability, pharmacokinetic properties and pharmacodynamic properties. The carrier materials preferably are water soluble or water dispersible and have wetting properties to increase the aqueous solubility and decrease the hydrophobicity of pharmaceutical compositions of the present invention. Where the composition is formulated as a tablet, the combination of carrier materials selected provides tablets that can exhibit, among other properties, improved dissolution and disintegration profiles, hardness, crushing strength, or friability properties.

### Diluents

The pharmaceutical compositions of the present invention optionally can comprise one or more diluents as a carrier material. Suitable diluents can include, either individually or in combination, such diluents as lactose USP; lactose USP, anhydrous; lactose USP, spray dried; starch USP; directly compressible starch; mannitol USP; sorbitol; dextrose monohydrate; microcrystalline cellulose NF; dibasic calcium phosphate dihydrate NF; sucrose-based diluents; confectioner's sugar; monobasic calcium sulfate monohydrate; calcium sulfate dihydrate NF; calcium lactate trihydrate granular NF; dextrates NF (e.g., Emdex™); Celutab™; dextrose (e.g., Cerelose™); inositol; hydrolyzed cereal solids such as the Maltrons™ and Mor-Rex™; amylose; Rexcel™; powdered cellulose (e.g., Elcema™); calcium carbonate; glycine; bentonite; polyvinylpyrrolidone; and the like. The present pharmaceutical compositions comprise one or more diluents in the range of about 5% to about 99%, preferably about 25% to about 90%, and more preferably about 40% to about 80%, of the total weight of the composition. The selected diluent or diluents preferably exhibit suitable compressibility and pre-compression flow properties. Microcrystalline cellulose (e.g. Avicel™ PH 101) and lactose, either individually or in combination are preferred diluents. The use of extragranular microcrystalline cellulose (that is, microcrystalline cellulose added to a wet granulated composition after the drying step) in addition to intragranular microcrystalline cellulose (that is, microcrystalline cellulose added to the composition during or before the wet granulation step) can be used to improve tablet hardness or disintegration time. Lactose, especially lactose monohydrate, is particularly preferred. Lactose typically provides pharmaceutical compositions having suitable release rates, stability, pre-compression flowability, and drying properties at a relatively low diluent cost.

### Disintegrants

The pharmaceutical compositions of the present invention optionally can comprise one or more disintegrants as a carrier material, particularly for tablet formulations. Suitable disintegrants can include, either individually or in combination, such disintegrants as starches; sodium starch glycolate; clays (such as Veegum™ HV); celluloses (such as purified cellulose, methylcellulose and sodium carboxymethylcellulose, and carboxymethylcellulose); alginates; pregelatinized corn starches (such as National™ 1551 and National™ 1550); crospovidone USP NF; gums (such as agar, guar, locust bean, Karaya™, pectin, and tragacanth). Disintegrants can be added at any suitable step during the preparation of the pharmaceutical composition, particularly prior to granulation or during the lubrication step prior to compression. The present pharmaceutical compositions comprise one or more disintegrants in the range of about 0.5% to about 30%, preferably about 1% to about 10%, and more preferably about 2% to about 6%, of the total weight of the composition. Croscarmellose sodium is a preferred disintegrant for tablet formulations, preferably in the range of about 1% to about 10%, preferably about 2% to about 6%, and more preferably about 5%, by weight of the composition.

### Binding Agents and Adhesives

The pharmaceutical compositions of the present invention optionally can comprise one or more binding agents or adhesives as a carrier material. Such binding agents and adhesives preferably impart sufficient cohesion to the powders to permit normal processing such as sizing, lubrication, compression and packaging, but still permit the tablet to disintegrate and the composition to dissolve upon ingestion. Suitable binding agents and adhesives include, either individually or in combination, such binding agents and adhesives as acacia; tragacanth; sucrose; gelatin; glucose; starch; cellulose materials such as, but not limited to, methylcellulose and sodium carboxymethylcellulose (e.g., Tylose™); alginic acid and salts of alginic acid; magnesium aluminum silicate; poly(ethylene glycol); guar gum; polysaccharide acids; bentonites; polyvinylpyrrolidone (povidone); polymethacrylates; hydroxypropylmethyl-cellulose (HPMC); hydroxypropyl cellulose (Klucel™); ethyl cellulose (Ethocel™); pregelatinized starch (such as National™ 1511 and Starch 1500). The present pharmaceutical compositions comprise one or more binding agents and/or adhesives in the range of about 0.5% to about 25%, preferably about 0.75% to about 15%, and more preferably about 1% to about 10%, of the total weight of the composition.

### Wetting Agents

Where it is desired to increase the aqueous solubility of a DSB salt of the present invention, the pharmaceutical compositions can optionally comprise one or more wetting agents as a carrier material, particularly for tablet formulations. Such wetting agents preferably maintain the DSB salt in solution and improve the bioavailability of the pharmaceutical composition. Suitable wetting agents include, either individually or in combination, such wetting agents as oleic acid; glyceryl monostearate; sorbitan monooleate; sorbitan monolaurate; triethanolamine oleate; polyoxyethylene sorbitan mono-oleate; polyoxyethylene sorbitan monolaurate; sodium oleate; and sodium lauryl sulfate. In some embodiments, wetting agents that are surfactants are preferred. In some embodiments, wetting agents that are anionic surfactants are preferred. The present pharmaceutical compositions comprise one or more wetting agents present at about 0.1% to about 15%, preferably about 0.25% to about 10%, and more preferably about 0.5% to about 5%, of the total weight of the composition. Sodium lauryl sulfate is a preferred wetting agent for tablet formulations. The compositions of the present invention preferably comprise sodium lauryl sulfate as the wetting agent at about 0.25% to about 7%, more preferably about 0.4% to about 4%, and still more preferably about 0.5 to about 2%, of the total weight of the composition.

### Lubricants

The pharmaceutical compositions of the present invention optionally comprise one or more lubricants or glidants as a carrier material. Suitable lubricants and/or glidants include, either individually or in combination, such lubricants and/or glidants as glyceryl behenate (Compritol™ 888); metalllic stearates (e.g., magnesium, calcium and sodium stearates); stearic acid; hydrogenated vegetable oils (e.g., Sterotex™); talc; waxes; Stearowet™; boric acid; sodium benzoate and sodium acetate; sodium chloride; DL-Leucine; polyethylene glycols (e.g., Carbowax™ 4000 and Carbowax™ 6000); sodium oleate; sodium benzoate; sodium acetate; sodium lauryl sulfate; sodium stearyl fumarate (Pruv™); and magnesium lauryl sulfate. The present pharmaceutical compositions comprise one or more lubricants at about 0.1% to about 10%, preferably about 0.2% to about 8%, and more preferably about 0.25% to about 5%, of the total weight of the composition. Magnesium stearate is a preferred lubricant used to reduce friction between the equipment and granulation during compression.

### Anti-Adherents or Glidants

The pharmaceutical compositions of the present invention optionally can comprise one or more anti-adherent agents or glidants as a carrier material. Suitable anti-adherents or glidants include, either individually or in combination, such anti-adherents as talc, cornstarch, Cab-O-Sil™, Syloid™, DL-Leucine, sodium lauryl sulfate, and metallic stearates. The present pharmaceutical compositions comprise one or more anti-adherents or glidants at about 0.1% to about 15%, preferably about 0.25% to about 10%, and more preferably about 0.5% to about 5%, of the total weight of the composition. Talc is a preferred anti-adherent or glidant agent used to reduce formulation sticking to equipment surfaces and also to reduce static in the blend. The compositions preferably comprise talc at about 0.1% to about 10%, more preferably about 0.25% to about 5%, and still more preferably about 0.5% to about 2%, of the total weight of the composition.

Other carrier materials (such as colorants, flavors and sweeteners) and modes of administration are known in the pharmaceutical art and can be used in the preparation of the pharmaceutical compositions of the present invention. Tablets can be coated or uncoated.

The individual pharmaceutically acceptable carrier materials described in the above embodiment optionally can be replaced with other suitable carrier materials if desired. Acceptable substitute carrier materials are chemically compatible both with the DSB salt of the present invention and with the other carrier materials.

Compositions within the scope of this invention include all compositions comprising at least one DSB salt according to the present invention in an amount effective to achieve its intended purpose. While individual needs vary, determination of optimal ranges of effective amounts of each component is within the skill of the art. Typical dosages of at least one DSB salt comprise about 0.05 to about 100 mg/kg body weight. In some embodiments, a useful dosage of one or more DSB salts comprises about 0.1 to about 100 mg/kg body weight of the active ingredient, preferably about 0.1 to about 20 mg/kg body weight of the active ingredient. In some embodiments, a more preferred dosage of one or more DSB salts comprises about 0.2 to about 10 mg/kg body weight. A useful dosage of one or more DSB salts comprises about 0.5 to about 5 mg/kg body weight. In some embodiments, the dosage of one or more DSB salts can comprise about 10 to about 100 mg/kg body weight.

Various amounts of one or more salts of the present invention can be administered according to the present invention. In some embodiments, about 10 mg to about 1000 mg of the active ingredients of one or more salts of the present invention can be administered once per day. In some embodiments, about 200 mg to about 800 mg of the active ingredient of one or more salts of the present invention can be administered once per day. In some embodiments, 100 mg, 200 mg, 300 mg, 400 mg, 500 mg or 600 mg of the active ingredient of one or more salts of the present invention is administered once per day. The amount of one or more salts administered per day is determined by the total amount of one or more salts administered in a 24 hour period. Thus, dosage regimens which instruct administration of one or more salts of the invention multiple times during a 24 hour period are within the scope of the invention if the cumulative amount administered during a 24 hour period is within the ranges listed above.

Therapeutic administration can also include prior, concurrent, subsequent or adjunctive administration of at least one additional salt of DSB according to the present invention or other therapeutic agent, such as an anti-viral or immune stimulating agent. In such an approach, the dosage of the second drug can be the same as or different from the dosage of the first therapeutic agent. In one embodiment of the present invention, the drugs are administered on alternate days in the recommended amounts of each drug.

Administration of a compound of the present invention can also optionally include previous, concurrent, subsequent or adjunctive therapy using immune system boosters or immunomodulators. In addition to the pharmacologically active compounds, a pharmaceutical composition of the present invention can also comprises at least one pharmaceutically acceptable excipient. In one embodiment, the composition, particularly those composition which can be administered orally, such as tablets, dragees, and capsules, and also composition which can be administered rectally, such as suppositories, as well as suitable solutions for administration by injection or orally, contain from about 0.01 to 99 percent of the active ingredient together with at least one excipient. In another embodiment, the composition comprises about 20 to about 75 percent of active compound(s), together with at least one excipient.

Pharmaceutical compositions of the present invention are manufactured in a manner which is itself known, for example, by means of conventional mixing, granulating, dragee-making, dissolving, or lyophilizing processes. Thus, pharmaceutical preparations for oral use can be obtained by combining the active compounds with solid excipients, if present, optionally grinding the resulting mixture, and processing the mixture of granules, after adding suitable auxiliaries, if desired or necessary, to obtain tablets or dragee cores.

The free acid of DSB can be obtained by the synthesis method described in U.S. Pat. No. 5,679,828.

Having now fully described this invention, it will be understood to those of ordinary skill in the art that the same can be performed within a wide and equivalent range of conditions, formulations and other parameters without affecting the scope of the invention or any embodiment thereof.

1. A disalt form of 3-O-(3′,3′-dimethylsuccinyl)betulinate, (“DSB”), comprising one equivalent of DSB and two equivalents of a counterion derived from a base that is (+)-arginine, choline hydroxide, diethylamine, or diethanolamine.

2. (canceled)

3. The compound of claim 1, wherein the counterion is (+)-arginine.

4. The compound of claim 3, wherein the compound is amorphous.

5. The compound of claim 3, wherein the compound is crystalline.

6. The compound of claim 4, wherein the compound is DSB-2 (+)-arginine Form I-a.

7. The compound of claim 5, wherein the compound is DSB-2 (+)-arginine Form II-a.

8. The compound of claim 7, characterized by an X-ray powder diffraction pattern exhibiting at least one diffraction peak corresponding to d-spacings of about 6.79, 5.97, 5.31, 4.65, 4.38, or 3.97 angstroms⁻¹.

9. The compound of claim 7, characterized by the X-ray powder diffraction pattern of FIG. 6.

10. A process of preparing the compound of claim 6 comprising the steps of:
(a) dissolving DSB free acid in ethanol to yield a DSB solution;
(b) dissolving (+)-arginine in water to yield an (+)-arginine solution; and,
(c) mixing two equivalents of the (+)-arginine solution with one equivalent of the DSB solution to yield DSB-2 (+)-arginine Form I-a.

11. The process of claim 10, further comprising the step of filtering off any remaining liquid to isolate DSB-2 (+)-arginine Form I-a.

12. A process of preparing the compound of claim 7, comprising the steps of:
(a) dissolving DSB-2 (+)-arginine Form I-a in 2,2,2-trifluoroethanol to yield a DSB solution; and,
(b) inducing crystallization of the DSB solution to yield DSB-2 (+)-arginine Form II-a.

13. The process of claim 12, further comprising the step of filtering off any remaining liquid to isolate the recrystallized DSB-2 (+)-arginine Form II-a.

14. The compound of claim 1, wherein the counterion is choline.

15. The compound of claim 14, wherein the compound is amorphous.

16. The compound of claim 14, wherein the compound is crystalline.

17. The compound of claim 16, wherein the compound is DSB-2 choline Form I-c.

18. The compound of claim 16, wherein the compound is DSB-2 choline Form II-c.

19. The compound of claim 16, wherein the compound is DSB-2 choline Form III-c.

20. The compound of claim 15, wherein the compound is DSB-2 choline Form IV-c.

21. A process of preparing the compound of claim 17, comprising the steps of:
(a) dissolving DSB free acid in ethanol to yield a DSB solution;
(b) dissolving choline hydroxide in water to yield a choline hydroxide solution; and,
(c) mixing two equivalents of the choline hydroxide solution with one equivalent of the DSB solution to yield DSB-2 choline Form I-c.

22. The process of claim 21, further comprising the step of filtering off any remaining liquid to isolate DSB-2 choline Form I-c.

23. The process of claim 21, further comprising the step of recrystallizing DSB-2 choline Form I-c from a solvent selected from the group consisting of methanol and ethanol to yield recrystallized DSB-2 choline Form I-c.

24. A process of preparing the compound of claim 18, comprising the steps of:
(a) dissolving DSB-2 choline Form I-c in a solvent selected from the group consisting of 1-propanol and 2-propanol to yield a DSB solution; and,
(b) inducing crystallization of the DSB solution to yield DSB-2 choline Form II-c.

25. The process of claim 24, further comprising the step of filtering off any remaining liquid to isolate DSB-2 choline Form II-c.

26. A process of preparing the compound of claim 19, comprising the steps of:
(a) dissolving DSB-2 choline Form I-c in a solvent selected from the group consisting of N,N-dimethylformamide and N,N-dimethylacetamide to yield a DSB choline disalt solution; and,
(b) inducing crystallization of the DSB solution to yield DSB-2 choline Form III-c.

27. The process of claim 26, further comprising the step of filtering off any remaining liquid to isolate DSB-2 choline Form III-c.

28. A process of preparing the compound of claim 15, comprising the steps of:
(a) dissolving DSB-2 choline Form I-c in a solvent selected from the group consisting of acetonitrile and water to yield a DSB solution; and,
(b) inducing crystallization of the DSB solution to yield DSB-2 choline Form IV-c.

29. The process of claim 28, further comprising the step of filtering off any remaining liquid to isolate DSB-2 choline Form IV-c.

30. The compound of claim 17, characterized by the X-ray powder diffraction pattern of FIG. 8.

31. The compound of claim 17, characterized by an X-ray powder diffraction pattern exhibiting at least one diffraction peak corresponding to d-spacings of about 8.61, 8.05, 7.48, 6.88, 6.17, 5.80, 5.54, 5.25, 4.89, 4.46, 4.13, 3.95, or 3.30 angstroms⁻¹.

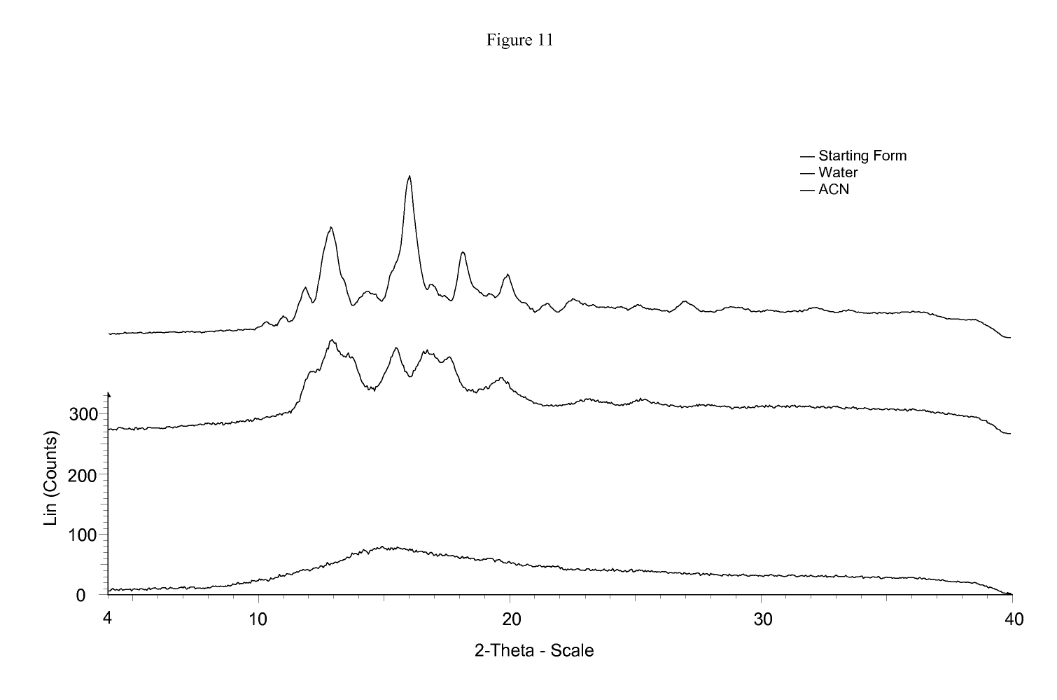

32. The compound of claim 18, characterized by the X-ray powder diffraction pattern of FIG. 11.

33. The compound of claim 18, characterized by an X-ray powder diffraction pattern exhibiting at least one diffraction peak corresponding to d-spacings of about 12.2, 15.8, 18.0, and 19.8 angstroms⁻¹.

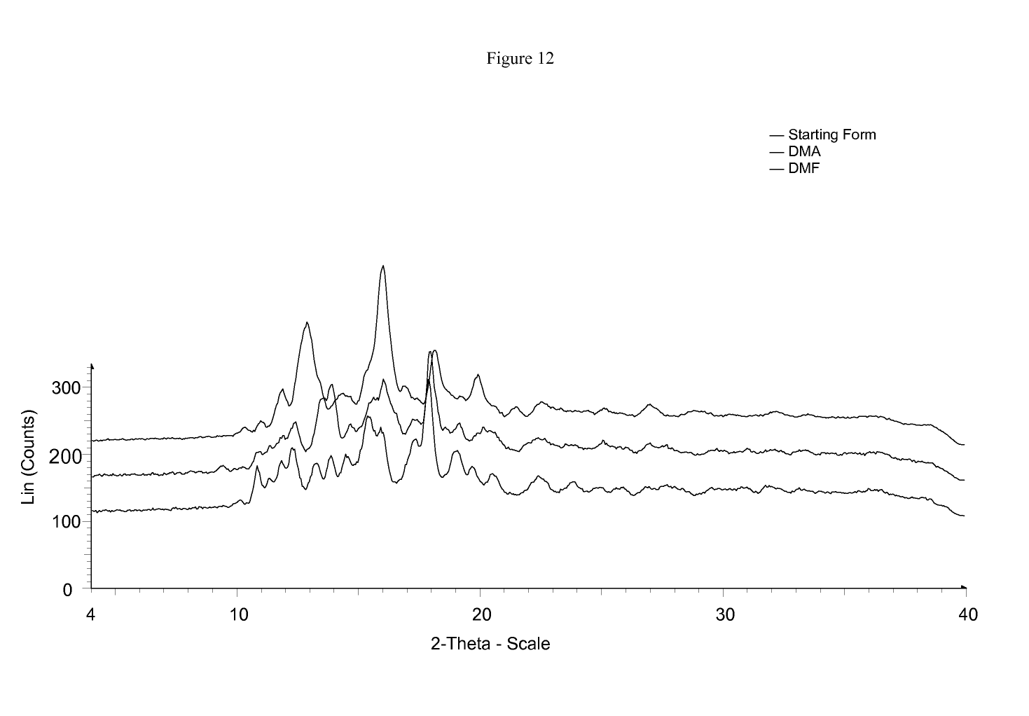

34. The compound of claim 19, characterized by the X-ray powder diffraction pattern of FIG. 12.

35. The compound of claim 19, characterized by an X-ray powder diffraction pattern exhibiting at least one diffraction peak corresponding to d-spacings of about 12.7, 16.0, and 18.0 angstroms⁻¹.

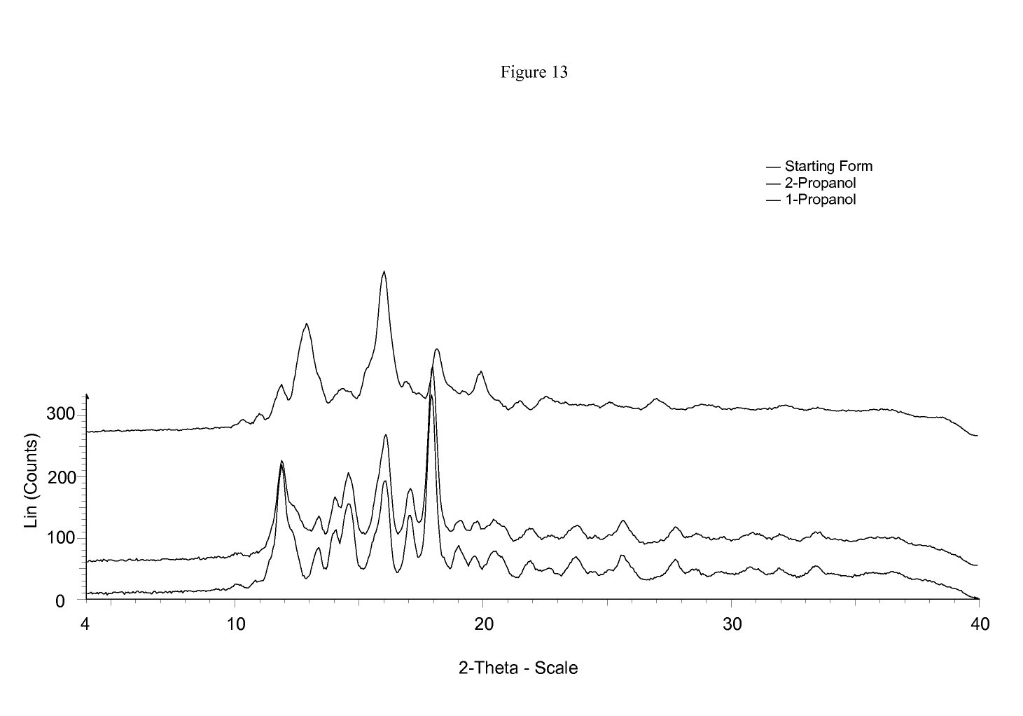

36. The compound of claim 20, characterized by the X-ray powder diffraction pattern of FIG. 13.

37. The compound of claim 20, characterized by an X-ray powder diffraction pattern exhibiting at least one diffraction peak corresponding to d-spacings of about 15.9 and 18.0 angstroms⁻¹.

38. The compound of claim 1, wherein the counterion is diethanolamine.

39. The compound of claim 38, wherein the compound is crystalline.

40. The compound of claim 39 wherein the compound is DSB-2 diethanolamine Form I-o.

41. The compound of claim 39, wherein the compound is DSB-2 diethanolamine Form II-o.

42. A process of preparing the compound of claim 40, comprising the steps of:
(a) dissolving DSB free acid in ethanol to yield a DSB solution;
(b) dissolving diethanolamine in water to yield an diethanolamine solution; and,
(c) mixing two equivalents of the diethanolamine solution with one equivalent of the DSB solution to yield DSB-2 diethanolamine Form I-o.

43. The process of claim 42, further comprising the step of filtering off any remaining liquid to isolate DSB-2 diethanolamine Form I-o.

44. The process of claim 42, further comprising the step of recrystallizing DSB-2 diethanolamine Form I-o from a solvent selected from the group consisting of methanol, ethanol, 1-propanol, 2-propanol, water, N,N-dimethylformamide, N,N-dimethylacetamide, acetone, ethyl acetate, and methylene chloride to yield recrystallized DSB-2 diethanolamine Form I-o.

45. A process of preparing the compound of claim 41, comprising the steps of:
(a) dissolving DSB-2 diethanolamine Form I-o in 2,2,2-trifluoroethanol to yield a DSB solution; and,
(b) inducing crystallization of the DSB solution to yield DSB-2 diethanolamine Form II-o.

46. The process of claim 45, further comprising the step of filtering off any remaining liquid to isolate DSB-2 diethanolamine Form II-o.

47. The compound of claim 40, characterized by the X-ray powder diffraction pattern of FIG. 15.

48. The compound of claim 40, characterized by an X-ray powder diffraction pattern exhibiting at least one diffraction peak corresponding to d-spacings of about 8.16, 6.64, 6.37, 5.54, 5.18, 4.49, 4.24, 3.91, 3.67, 3.44, 3.39, 3.13, 2.90, 2.70, 2.55, or 2.34 angstroms⁻¹.

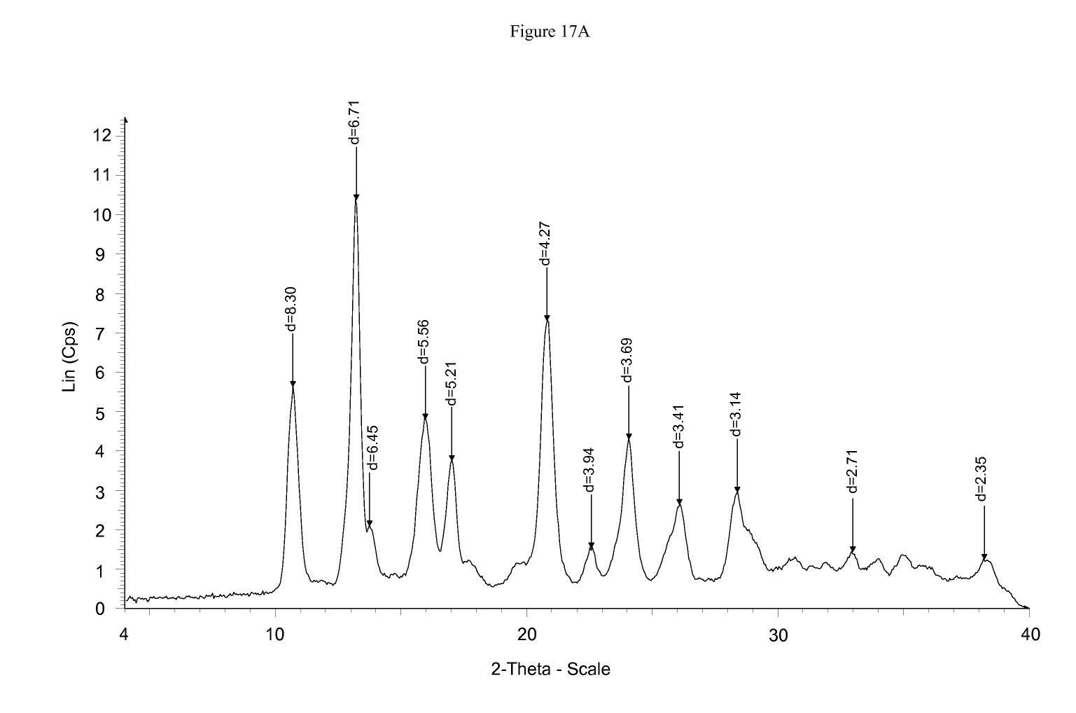

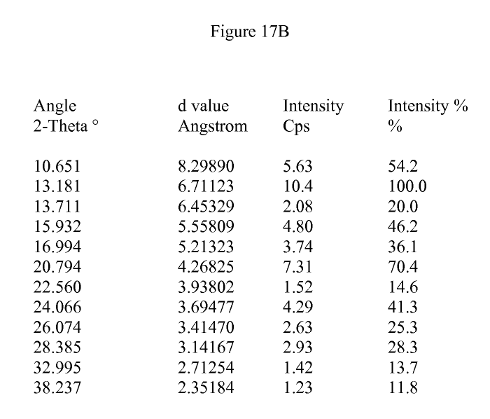

49. The compound of claim 41, characterized by the X-ray powder diffraction pattern of FIG. 17.

50. The compound of claim 41, characterized by an X-ray powder diffraction pattern exhibiting at least one diffraction peak corresponding to d-spacings of about 8.30, 6.71, 6.45, 5.56, 5.21, 4.27, 3.93, 3.69, 3.41, 3.14, 2.71, or 2.35 angstroms⁻¹.

51. The compound of claim 1, wherein the counterion is diethylamine.

52. The compound of claim 51, wherein the compound is crystalline.

53. The compound of claim 52, wherein the compound is DSB-2 diethylamine Form I-y.

54. The compound of claim 52, wherein the compound is DSB-2 diethylamine Form II-y.

55. The compound of claim 52, wherein the compound is DSB-2 diethylamine Form III-y.

56. The compound of claim 52, wherein the compound is DSB-2 diethylamine Form IV-y.

57. A process of preparing the compound of claim 53, comprising the steps of:
(a) dissolving DSB free acid in ethanol to yield a DSB solution;
(b) dissolving diethylamine in water to yield a diethylamine solution; and,
(c) mixing two equivalents of the diethylamine solution with one equivalent of the DSB solution to yield DSB-2 diethylamine Form I-y.

58. The process of claim 57, further comprising the step of filtering off any remaining liquid to isolate DSB-2 diethylamine Form I-y.

59. The process of claim 57, further comprising the step of recrystallizing DSB-2 diethylamine Form I-y in a solvent selected from the group consisting of ethanol, 2,2,2-trifluoroethanol, 1-propanol, 2-propanol, water, and acetone to yield DSB-2 diethylamine Form I-y.

60. A process of preparing the compound of claim 54, comprising the steps of:
(a) dissolving DSB-2 diethylamine Form I-y in methylene chloride to yield a DSB solution; and,
(b) inducing crystallization of the DSB solution to yield DSB-2 diethylamine Form II-y.

61. The process of claim 60, further comprising the step of filtering off any remaining liquid to isolate DSB-2 diethylamine Form II-y.

62. A process of preparing the compound of claim 55, comprising the steps of:
(a) dissolving DSB-2 diethylamine Form I-y in methanol to yield a DSB solution; and,
(b) inducing crystallization of the DSB solution to yield DSB-2 diethylamine Form III-y.

63. The process of claim 62, further comprising the step of filtering off any remaining liquid to isolate DSB-2 diethylamine Form III-y.

64. A process of preparing the compound of claim 56, comprising the steps of:
(a) dissolving DSB-2 diethylamine Form I-y in a solvent selected from the group consisting of N,N-dimethylformamide and ethyl acetate to yield a DSB solution; and,
(b) inducing crystallization of the DSB solution to yield DSB-2 diethylamine Form IV-y.

65. The process of claim 61, further comprising the step of filtering off any remaining liquid to isolate DSB-2 diethylamine Form IV-y.

66. The compound of claim 53 characterized by the X-ray powder diffraction pattern of FIG. 20.

67. The compound of claim 53, characterized by an X-ray powder diffraction pattern exhibiting at least one diffraction peak corresponding to d-spacings of about 9.37, 7.78, 7.25, 6.63, 6.20, 5.59, 5.24, 5.07, 4.86, 4.68, 4.53, 4.20, 3.99, 3.85, 3.70, 3.39, 3.25, 3.03, or 2.36 angstroms⁻¹.

68. The compound of claim 54, characterized by the X-ray powder diffraction pattern of FIG. 24.

69. The compound of claim 54, characterized by an X-ray powder diffraction pattern exhibiting at least one diffraction peak corresponding to d-spacings of about 8.76, 7.81, 7.19, 6.79, 6.01, 5.65, 5.27, 4.63, 4.08, 3.95, 3.75, or 3.43 angstroms⁻¹.

70. The compound of claim 55, characterized by the X-ray powder diffraction pattern of FIG. 25.

71. The compound of claim 55, characterized by an X-ray powder diffraction pattern exhibiting at least one diffraction peak corresponding to d-spacings of about 8.52, 7.81, 7.14, 6.81, 5.98, 5.67, 5.32, 4.88, 4.66, 4.39, 3.98, 3.44, 3.26, 2.91, or 2.31 angstroms⁻¹.

72. The compound of claim 56, characterized by the X-ray powder diffraction pattern of FIG. 23.

73. The compound of claim 56, characterized by an X-ray powder diffraction pattern exhibiting at least one diffraction peak corresponding to d-spacings of about 10.37, 11.32, 12.40, 12.99, 14.79, 15.62, 16.65, 18.15, 19.01, 20.19, 22.34, 25.88, 27.34, 30.71, 38.95 angstroms⁻¹.

74. A method for the prevention or treatment of HIV-1, comprising administering to a patient in need of such prevention or treatment a therapeutically effective amount of the compound of claim 5, and a pharmaceutically acceptable excipient.

75. A method for the prevention or treatment of HIV-1, comprising administering to a patient in need of such prevention or treatment, a therapeutically effective amount of the compound of claim 16, and a pharmaceutically acceptable excipient.

76. A method for the prevention or treatment of HIV-1, comprising administering to a patient in need of such prevention or treatment, a therapeutically effective amount of the compound of claim 39, and a pharmaceutically acceptable excipient.

77. A method for the prevention or treatment of HIV-1, comprising administering to a patient in need of such prevention or treatment, a therapeutically effective amount of the compound of claim 52, and a pharmaceutically acceptable excipient.
